(19) United States
(12) Patent Application Publication
(10) Pub. No.: US 2018/0117249 A1
(43) Pub. Date: May 3, 2018

(54) Warburg Effect Targeted Chemotherapy Apparatus

(71) Applicant: Cure Cancer Worldwide LLC, Tucson, AZ (US)

(72) Inventors: Michael E. Pennington, Tucson, AZ (US); Linda Meade-Tollin, Tucson, AZ (US); Tom C. Tsang, Tucson, AZ (US)

(73) Assignee: Cure Cancer Worldwide LLC, Tucson, AZ (US)

(21) Appl. No.: 15/825,613

(22) Filed: Nov. 29, 2017

Related U.S. Application Data
(63) Continuation-in-part of application No. 13/911,977, filed on Jun. 6, 2013.
Application No. 13/911,977 is a continuation of application No. PCT/CN2011/002035, filed on Dec. 6, 2011.
(60) Provisional application No. 62/428,440, filed on Nov. 30, 2016.
(60) Provisional application No. 61/420,208, filed on Dec. 6, 2010.

(51) Int. Cl. A61M 5/172; A61B 5/145; A61B 5/0402; A61B 5/00; A61K 38/28; A61K 31/7004; A61K 45/06; A61P 31/00; A61P 35/04; A61P 3/10; A61P 3/08; A61K 9/00; A61M 5/14; A61M 5/168; A61M 5/142

(52) U.S. Cl. CPC A61M 5/1723; A61M 2205/18; A61B 5/0402; A61B 5/4839; A61K 38/28; A61K 31/7004; A61K 45/06; A61P 31/00; A61P 35/04; A61P 3/10; A61P 3/08; A61K 9/0019; A61M 5/1413; A61M 5/1409; A61M 5/16831; A61M 5/142; A61M 5/16827; A61M 2205/50; A61M 2230/201; A61M 2230/04; A61M 2230/30; A61M 2205/36; A61M 2205/3569; A61M 2205/52; A61M 2205/502; A61M 2205/581; A61M 2205/587; A61M 2205/6072; A61M 2205/3337; A61M 2205/505; A61B 5/14532

(57) ABSTRACT

A system for chemotherapy delivery comprises a plurality of slots to receive a corresponding one of a plurality of cartridges; a plurality of pumps, wherein each of the plurality of pumps is configured to be connected to the corresponding one of the plurality of cartridges, and the plurality of pumps are configured to pump at least one drug contained in at least one of the plurality of cartridges to a patient according to a treatment protocol; a blood glucose sensor communicatively coupled to the plurality of pumps, and configured to measure a blood glucose level of the patient; a processor connected to the plurality of pumps and the blood glucose sensor and configured to adjust a delivery property of the at least one drug according to the measured blood glucose level of the patient.

### CROSS-REFERENCE TO RELATED APPLICATIONS

This application claims the benefit of U.S. Provisional Application No. 62/428,440, entitled “WARBURG EFFECT TARGETED CHEMOTHERAPY APPARATUS” filed Nov. 30, 2016, and is a Continuation-in-Part of U.S. application Ser. No. 13/911,977, entitled “METHODS OF METABOLIC TARGETING CANCER CELLS USING CHEMO- AND IMMUNOTHERAPY FOR TREATING CANCER” filed Jun. 6, 2013, which is a Continuation of International Application No. PCT/CN2011/002035, entitled “METHODS OF METABOLIC TARGETING CANCER CELLS USING CHEMO- AND IMMUNOTHERAPY FOR TREATING CANCER,” filed Dec. 6, 2011, which claims the benefit of U.S. Provisional Application 61/420,208, entitled “METHODS OF METABOLIC TARGETING CANCER CELLS USING CHEMO- AND IMMUNOTHERAPY FOR TREATING CANCER,” filed Dec. 6, 2010, the contents of which are hereby incorporated by reference in their entirety, as if fully set forth herein.

### TECHNICAL FIELD

The application relates to Chemotherapy Apparatus, and more particular, to Warburg Effect targeted chemotherapy apparatus.

### BACKGROUND

Cancer is a disease of altered metabolism as much as it is a disease of uncontrolled cell growth. Targeting of metabolic pathways in cancer cells has a long history with many of the current chemotherapeutic drugs targeting a few of those pathways. In recent years, additional metabolic pathways have come of interest to target, namely cancer's reliance on glucose for energy. This altered metabolism in cancer is best utilized in PET scan technology, with sensitivity rates over 90%, making this altered metabolism nearly a universal trait of cancer.

Cancer is a leading cause of death worldwide. In the United States alone, over 1.3 million people are diagnosed with cancer each year and over 500,000 die. Due to the high incidence and mortality rate, research efforts have focused on improving treatment options for those who are diagnosed with cancer, but a cure has been elusive, especially in later stages of the disease.

Treatment options are determined by the type and stage of the cancer and the patient's overall health. Several modalities of treatment are available, including surgery, chemotherapy, radiation therapy, targeted therapy and immunotherapy. Primary tumors in early stages are sometimes treated by surgery followed by radiation therapy, but in general, most cancers involve treatment with chemotherapy.

Chemotherapy drugs are administered systemically and attack all cells of the body, not just cancer cells. Some chemotherapy drugs are used alone for treating cancer, but often several drugs may be combined, known as combination chemotherapy. Further, chemotherapy is often used together with other modalities of treatment such as surgery, radiation therapy, targeted therapy and immunotherapy.

Because chemotherapy drugs are usually given at the maximum tolerated dose, frequent and dramatic toxicities result that compromise the quality of life and the immune response toward opportunistic infection and toward the cancer itself. These toxicities manifest themselves as side effects such as nausea, hair loss (alopecia), hematopoietic toxicity, decreased mobilization of hematopoietic progenitor cells from bone marrow into the peripheral blood, anemia, myelosuppression, pancytopenia, thrombocytopenia, neutropenia, lymphopenia, leucopenia, stomatitis, esophagitis, heart damage, nervous system damage, lung damage, reproductive system damage, liver damage, kidney and urinary system damage, fatigue, constipation, diarrhea, loss of appetite, headache and muscle pain. These side effects often limit the dose of the chemotherapy agents that can be administered and the frequency at which they can be given.

Acute myelosuppression as a consequence of chemotherapy is well recognized as a dose-limiting factor in cancer treatment. Although other normal tissues may also be adversely affected, bone marrow is particularly sensitive to proliferation-specific treatments such as chemotherapy or radiation therapy. Repeated or high dose cycles of chemotherapy may result in severe stem cell depletion leading to long-term immune suppression or exhaustion. Immune suppression and other side effects often limit the dose or frequency at which treatments may be given, interfere with other treatments that are used in combination with chemotherapy, and otherwise cause interruption of cancer treatments and allow the disease to progress.

Therefore, there is a need for improved therapeutic methods for treating cancer that decrease side effects of chemotherapy and increase the efficacy of chemotherapy, by itself and when used in combination with other modalities of cancer treatment.

### SUMMARY OF THE INVENTION

According to a first aspect of the disclosure, a system for chemotherapy delivery, comprising a plurality of slots, wherein each of the plurality of slots is configured to receive a corresponding one of a plurality of cartridges; a plurality of pumps, wherein each of the plurality of pumps is configured to be connected to the corresponding one of the plurality of cartridges, and the plurality of pumps are configured to pump at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively; a blood glucose sensor communicatively coupled to the plurality of pumps, and configured to measure a blood glucose level of the patient; a processor connected to the plurality of pumps and the blood glucose sensor and configured to adjust delivery property of the at least one drug according to the measured blood glucose level of the patient; and wherein the plurality of pumps are further configured by the processor to adjust pumping the at least one drug according to the adjusted delivery property.

According to another aspect of the disclosure, a method for chemotherapy delivery, comprising: receiving, by each of a plurality of slots, a corresponding one of a plurality of cartridges; pumping, by a plurality of pumps each connected to the corresponding one of the plurality of cartridges, at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively; measuring, by a blood glucose sensor communicatively coupled to the plurality of pumps, a blood glucose level of the patient; adjusting, by a processor connected to the plurality of pumps and the blood glucose sensor, delivery property of the at least one drug according to the measured blood glucose level of the patient; and adjusting, by the plurality of pumps, pumping the at least one drug according to the adjusted delivery property.

According to a third aspect of the disclosure, a computer readable storage medium, storing instructions when executed by a processor, cause the computer to perform operations comprising: controlling, a plurality of pumps each connected to a corresponding one of a plurality of cartridges to pump at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively; controlling a blood glucose sensor communicatively coupled to the plurality of pumps to measure a blood glucose level of the patient; adjusting delivery property of the at least one drug according to the measured blood glucose level of the patient; and controlling the plurality of pumps to adjust pumping the at least one drug according to the adjusted delivery property.

Methods for treating cancer comprising administering a metabolic targeting chemo-immunotherapy regimen are provided herein. In one embodiment, the metabolic targeting chemo-immunotherapy regimen comprises administering a therapeutically effective dose of one or more immunologic agents to stimulate an immune response in a subject having cancer; reducing the patient's blood glucose level; and administering a therapeutically effective dose of one or more chemotherapeutic agents. The blood glucose level may be reduced by fasting, administering a dose of insulin, or a combination thereof.

The one or more immunologic agents are selected from the group consisting of vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies. Examples of such agents are discussed in detail below.

In another embodiment, the metabolic targeting chemo-immunotherapy regimen comprises administering an initial therapeutically effective dose of a therapeutic antibody or functional fragment thereof to target a population of cancer cells and to stimulate an immune response in a subject having cancer; reducing the patient's blood glucose level by fasting and/or administering a dose of insulin; and administering a therapeutically effective dose of one or more chemotherapeutic agents. The blood glucose level may be reduced by fasting, administering a dose of insulin, or a combination thereof.

In another embodiment, the metabolic targeting chemo-immunotherapy regimen comprises the steps of a) administering an initial therapeutically effective dose of one or more therapeutic antibodies to a subject having cancer to stimulate an immune response; b) fasting the subject overnight; c) administering an effective dose of insulin to the subject to reduce the subject's blood glucose level; and d) administering a therapeutically effective dose of one or more chemotherapeutic agents.

When the methods described herein include administering a therapeutic antibody or functional fragment thereof, said selected from the group consisting of alemtuzumab, bevacizumab, cetuximab, edrecolomab, gemtuzumab, ibritumomab tiuxetan, panitumumab, rituximab, tositumomab, and trastuzumab. In one embodiment, the method may further comprise administering one or more booster doses of the one or more therapeutic antibodies. The one or more booster doses may be administered at any interval, including, but not limited to, an interval of two weeks.

The one or more chemotherapeutic agents are selected from the group consisting of alkylating agents, antimetabolites, anti-tumor antibiotics, topoisomerase inhibitors, mitotic inhibitors hormone therapy, glycolysis inhibitors, targeted therapeutics and immunotherapeutics.

The methods for metabolic targeting chemo-immunotherapy described herein are used for treating a cancer selected from the group consisting of bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer.

### BRIEF DESCRIPTION OF THE DRAWINGS

Non-limiting and non-exhaustive embodiments of the present invention are described with reference to the following figures, wherein like reference numerals refer to like parts throughout the various views unless otherwise specified.

FIG. 1 is a schematic diagram illustrating comparisons of conventional chemotherapy treatment versus Warburg Effect Target Chemotherapy on cancer stem cells, according to the embodiments described herein.

FIG. 2 is a diagram illustrating pharmacokinetics of hypoglycemic glucose clamp metabolic targeted chemotherapy protocol according to an embodiment of the invention.

FIG. 3 is a diagram for a glucose clamp procedure done on mice with flank tumors.

FIG. 4 is results from two different glucose clamp plus chemotherapy procedures performed on mice with different tumor types derived from different human cancer cell lines.

FIG. 5 is a diagram for a system for chemotherapy delivery according to an embodiment of the invention.

FIG. 6 is a block diagram illustrating a system for chemotherapy delivery according to an embodiment of the invention.

FIG. 7 is a block diagram for a system for chemotherapy delivery according to another embodiment of the invention.

FIG. 8 is a system logic diagram for chemotherapy delivery according to an embodiment of the invention.

FIG. 9 is a high-level extent diagram showing an example of the architecture of the device for chemotherapy delivery according to an embodiment of the invention.

FIG. 10 is a flow diagram illustrating an example of method of chemotherapy delivery according to an embodiment of the invention.

FIG. 11 is a flow diagram illustrating an example of method of chemotherapy delivery according to another embodiment of the invention.

FIG. 12 is a schematic showing multi-step carcinogenesis of cancer stem cells (CSC) through cell fusion.

FIG. 13 is a schematic diagram illustrating comparisons of typical chemotherapy treatment versus metabolic targeted chemo-immunotherapy on cancer stem cells, according to the embodiments described herein.

FIG. 14 shows representative PET/CT scans for an exemplar patient (Patient 4) receiving the metabolic targeting chemo-immunotherapy described in the embodiments herein.

FIG. 15 shows a lung lesion from Patient 4.

### DETAILED EMBODIMENTS

The embodiments set forth below represent the necessary information to enable those skilled in the art to practice the embodiments, and illustrate the best mode of practicing the embodiments. Upon reading the following description in light of the accompanying figures, those skilled in the art will understand the concepts of the disclosure and will recognize applications of these concepts that are not particularly addressed here. It should be understood that these concepts and applications fall within the scope of the disclosure and the accompanying claims.

The purpose of terminology used herein is only for describing embodiments and is not intended to limit the scope of the disclosure. Where context permits, words using the singular or plural form may also include the plural or singular form, respectively.

As used herein, unless specifically stated otherwise, terms such as “processing,” “computing,” “calculating,” “determining,” “displaying,” “generating,” or the like, refer to actions and processes of a computer or similar electronic computing device that manipulates and transforms data represented as physical (electronic) quantities within the computer's memory or registers into other data similarly represented as physical quantities within the computer's memory, registers, or other such storage medium, transmission, or display devices.

As used herein, terms such as “connected,” “coupled,” or the like, refer to any connection or coupling, either direct or indirect, between two or more elements. The coupling or connection between the elements can be physical, logical, or a combination thereof. References in this description to “an embodiment,” “one embodiment,” or the like, mean that the particular feature, function, structure or characteristic being described is included in at least one embodiment of the present disclosure. Occurrences of such phrases in this specification do not necessarily all refer to the same embodiment. On the other hand, the embodiments referred to also are not necessarily mutually exclusive.

As used herein, terms such as “cause” and variations thereof refer to either direct causation or indirect causation. For example, a computer system can “cause” an action by sending a message to a second computer system that commands, requests, or prompts the second computer system to perform the action. Any number of intermediary devices may examine and/or relay the message during this process. In this regard, a device can “cause” an action even though it may not be known to the device whether the action will ultimately be executed.

Note that in this description, any references to sending or transmitting a message, signal, etc. to another device (recipient device) means that the message is sent with the intention that its information content ultimately be delivered to the recipient device; hence, such references do not mean that the message must be sent directly to the recipient device. That is, unless stated otherwise, there can be one or more intermediary entities that receive and forward the message/signal, either “as is” or in modified form, prior to its delivery to the recipient device. This clarification also applies to any references herein to receiving a message/signal from another device; i.e., direct point-to-point communication is not required unless stated otherwise herein.

As used herein, unless specifically stated otherwise, the term “or” can encompass all possible combinations, except where infeasible. For example, if it is stated that data can include A or B, then, unless specifically stated otherwise or infeasible, the data can include A, or B, or A and B. As a second example, if it is stated that data can include A, B, or C, then, unless specifically stated otherwise or infeasible, the data can include A, or B, or C, or A and B, or A and C, or B and C, or A and B and C.

Localized Glucose Concentrations in Cancer Tissue

A recent study in Cancer Research looked at different metabolites in freshly frozen cancerous and normal tissues post-surgery. This study found glucose levels in cancerous tissues were 3 to 12 times less than healthy tissue. (Hirayama, et al., 2009) Due to the high glycolysis rate the cancer tissue is already depleted of glucose. This will of course be more pronounced in poorly vascularized ischemic tissue.

Glycolysis Effects on the Redox Status of Cellular Survival and Drug Resistance

Cancer cells often survive in an oxidative stressed environment. Many cancer drugs work through an increase in oxidative stress. Apoptosis requires an oxidative state within the cell to trigger and initiate the cascade that leads to cell death. Cancer cells are able to cope with the oxidative stress and avoid apoptosis through reduction via glutathione, the main cellular antioxidant. (Vaughn, et al., 2008) Oxidized glutathione is reconstituted via glutathione reductase using NADPH as an electron donor and producing NADP⁺. NADP⁺ is converted to NADPH in the pentose phosphate pathway, one of the two pathways which metabolize glucose. The other pathway, standard glycolysis, can be quickly shifted over to the pentose phosphate pathway upon the cell receiving oxidative stress, such as that delivered by drugs. (Patra, et al., 2014)

Cancer cells have a high capacity to metabolize glucose due to over expression of glucose transporters and metabolic enzymes. This high level of glycolysis leads to a buffering effect against oxidative stress via the recovery of oxidized glutathione. Many cancer drugs damage cancer cells through oxidative stress and high rates of glycolysis convey drug resistance to these drugs such as daunorubicin (Cao. Et al. 2007), adriamycin and paclitaxel (Maschek, et al., 2004), prednisolone (Ingrid et al., 2013), sorafenib (Tesori, 2015), and 5-fluorouracil (Shin, et al, 2009). Export of cisplatin by ABC transporters from cancer cells has been shown to be dependent on glutathione (Chen and Kuo, 2010).

Inhibition of Glycolysis

Inhibition of glycolysis is the core strategy to embodiments of the disclosure. Cancer cells which are unable to use glycolysis for energy generally become starved and rely on glutamine and autophagocytosis for energy. This can lead to cell necrosis. By limiting the level of blood glucose cancer cells quickly become depleted of their primary energy source. This will have many effects including the before mentioned necrosis and rate limited energy processes such as ATP-binding cassette transporters (ABC transporters). (Doyle, et al., 1998)

There are several investigational agents currently under study for glucose inhibition. One of these agents is 2-deoxyglucose (2DG) which shown effectiveness in the laboratory setting and has under gone clinical trials. 2DG being a glucose derivative is unable to be phosphorylated by hexokinase and can inhibit hexokinase activity. 2DG did however fail in the clinic. One of the main failure points was 2DG induced hyperglycemia. Cancer cells with elevated levels of Glucose transporter 1 (GLUT1 transporter) and hexokinase are able to survive in the enriched glucose environment despite the partial hexokinase inhibition. In addition, the 2DG can lead to irregular Electrocardiograph (ECG) heart rhythms. (Raez, et al., 2013).

One of a strategy of at least one embodiment for glycolysis inhibition is not to inhibit the common enzymes and glucose transporters, which are over expressed in cancer and requires enough inhibitor to completely disrupt and kill normal cells, but rather to inhibit glucose availability, which cancer cells require in larger quantities than normal cells to maintain their reductive state.

Warburg Effect Targeted Chemotherapy

Warburg effect targeted chemotherapy (WETC) uses insulin to induce hypoglycemia. Insulin does not interfere with any glycolysis enzymes. Insulin does however reduce overall blood glucose levels. Cancer tissue under normal blood glucose levels is already depleted of glucose which makes cancer tissue more sensitive to hypoglycemia. Additionally insulin induced hypoglycemia is a rapidly reversible process by giving intravenous glucose increasing the safety profile of this therapy.

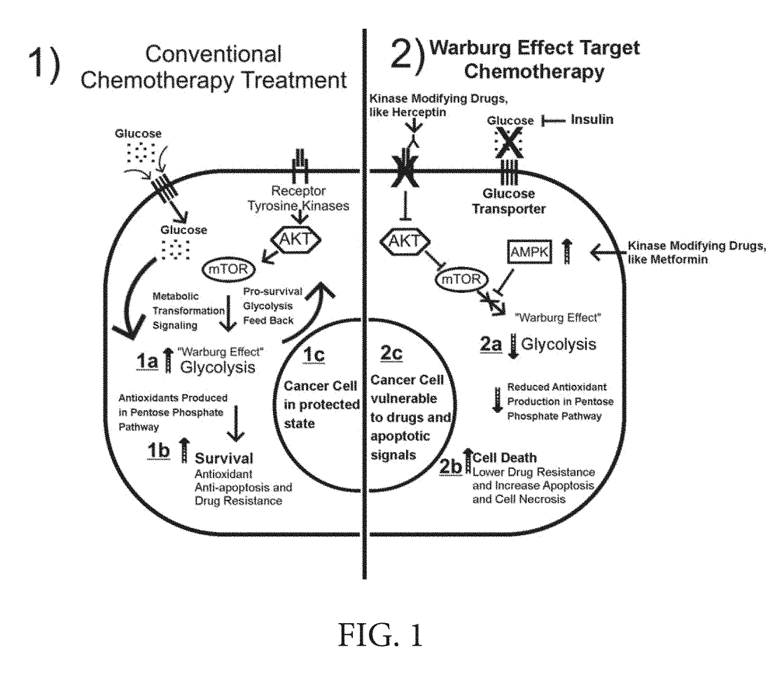

FIG. 1 is a schematic diagram illustrating comparisons of conventional chemotherapy treatment versus Warburg Effect Target Chemotherapy on cancer stem cells, according to the embodiments described herein. Warburg effect targeted chemotherapy, as shown in FIG. 1, is fundamentally different than conventional chemotherapy. Panel 1) (left part) of FIG. 1 shows the conventional chemotherapy treatment, which features 1a) Under normal blood glucose levels cancer cells are fully feed through the use of glucose for energy resulting in the necessary ATP for drug resistance. 1b) A high rate of glycolysis results in additional drug resistance and 1c) this resistance protects the cancer cells DNA from DNA damaging drugs. Panel 2) (right part) of FIG. 1 shows the Warburg Effect Target Chemotherapy, which features 2a) Under hypoglycemia cancer cells quickly deplete locally available glucose and enter in a metabolic crisis, resulting in lower ATP levels for normal cancer cell functions such as ABC transporters. 2b) A lower rate of glycolysis results in drug sensitivity. 2c) Hyperglycemic cancer cells' DNA is unprotected from DNA damaging chemotherapy drugs resulting in increased cancer cell death.

Safety of Warburg Effect Targeted Chemotherapy—Xi'an, China

Cure Cancer Worldwide in conjunction with several hospitals in China have been treating patients using a combination of insulin induced hypoglycemia in combination with standard chemotherapy treatments, generally given multiple times in a 3 week window but at 10% the standard dose. The safety of these treatments has been evaluated as well as the side effects.

Insulin-Induced Hypoglycemia Safety Profile

| The initial | | Time | Insulin | | |
|---|---|---|---|---|---|
| levels of | Hypoglycemia | Interval of | injection | | |
| blood glucose | range | Hypoglycemia | times | Comas | Deaths |
| (mmol/l) | (mmol/l) | (m) | (n) | (n) | (n) |
| 3.86-7.95 | 1.0-3.4 | 5-25 | 950 | 0 | 0 |

| | |
|---|---|
| Conclusion | safe |

Insulin Induces Retention of 5-Fluoro-Uricil in Cancer Cells

5-fluorourcil (5-FU) is a common drug used in chemotherapy and was developed in the 1950s. 5-FU is transported into cells via nucleoside transporters and is from there metabolized into several metabolites, some of which block RNA and DNA synthesis. One of the metabolites, 5-FdUMP, along with 5,10-methylenetetrahydrofolate (5,10-CH2-THF), forms a complex with thymidylate synthase (TS) which inactivates TS's function. The inactivation of TS causes insufficient thymidine for DNA synthases and repair. This makes synthesis phase (S-phase) cells especially sensitive to 5-FU. 5,10-CH2-THF is a downstream metabolite of folate, who's intracellular concentration is increased by insulin via decrease activity of the folate export mechanism. Levofolinic acid (leucovorin) is often given in conjunction with 5-FU to provide the folate precursor for 5,10-CH2-THF production necessary for 5-FU's inhibitory complex with thymidylate synthase.

A published study in 2007 (Zou et. al. Acta pharmacologica sinica 28.5 (2007): 721-730.) showed pretreatment with insulin several hours before addition of 5-FU resulted in greater inhibition of cell growth and higher percentage of apoptotic cell populations when compared to cells treated with 5-FU but without insulin treatment. They also showed an increase in the percent of S-phase cells with treatment of insulin. They claimed enhanced uptake of 5-FU by cells treated with insulin but did so indirectly. 5-FU concentrations in cell culture media was decreased when cells were treated with insulin and there was an increase in the 5-FdUMP/5,10-CH2-THF/TS complex. They attributed these observations to increased uptake but did not make the link between increase metabolism of folate and incorporation into the TS complex.

While the pentose phosphate pathway is the main source of NADPH in most cancer cells, folate metabolism can also produce NADPH from NADP⁺. Inhibition of the folate pathway, such as the use of 5-FU, along with hypoglycemia induced glucose deprivation, adds a combinatorial effect by furthering reducing available glutathione.

Pharmacokinetics of Warburg Effect Targeted Chemotherapy

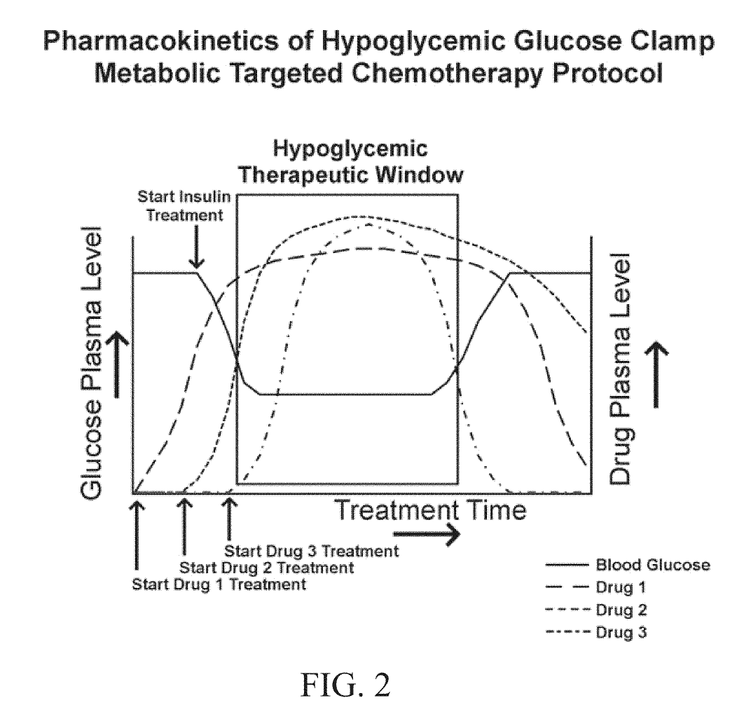

Precise timing is necessary when combining anti-cancer drugs and hypoglycemia. The drugs need to be at their peak pharmacological effectiveness during the “Hypoglycemic Therapeutic Window”, see FIG. 2. FIG. 2 is a diagram illustrating pharmacokinetics of hypoglycemic glucose clamp metabolic targeted chemotherapy protocol according to an embodiment of the invention. As shown in FIG. 2, drugs can be given before, during or after induction of hypoglycemia dependent upon their pharmacological profile so their corresponding peak correlates with that of the hypoglycemic therapeutic window.

Glucose Clamp as a Means to Induce Hypoglycemia

The glucose clamps technique was first developed in 1979 and is a means of delivering insulin and glucose intravenously to precisely control blood glucose levels. This technique is used in the field of diabetes to diagnose and develop new drugs for diabetes. It has been used safely to induce hyperglycemia and hypoglycemia as it provides a finely controlled way to induce changes in blood glucose levels. Hypoglycemia has been shown to be safe at 3.0 mmol/L for up to two hours although with altered ECG readings. (Laitinen, et al., 2008)

Preclinical Animal Trials Utilizing Glucose Clamps to Create the Hypoglycemic Therapeutic Window

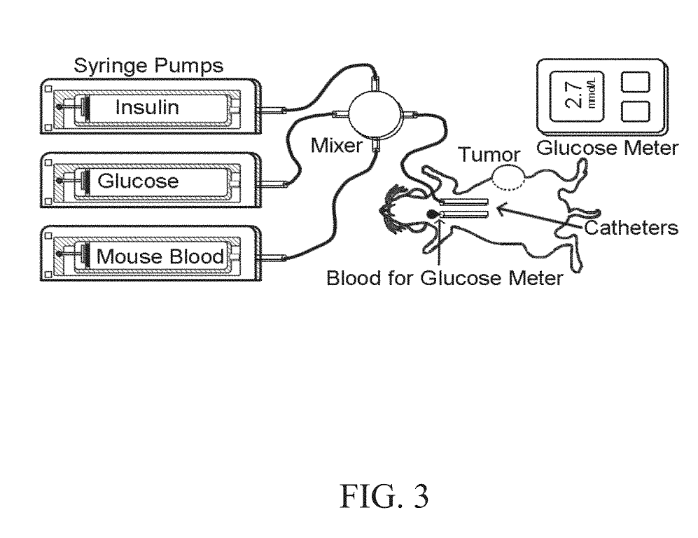

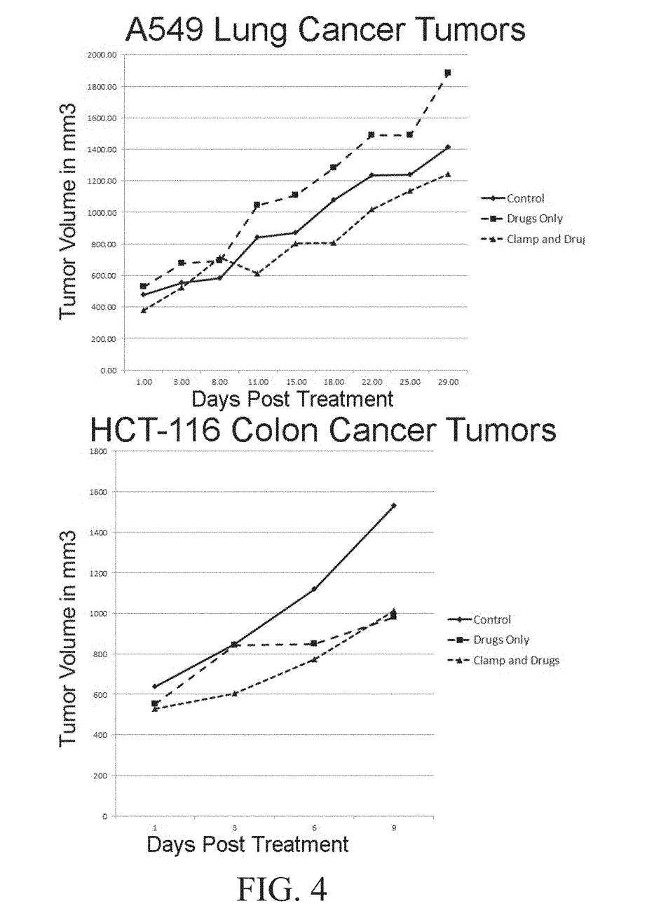

The glucose clamp technique has been well developed in animals models for the purpose diabetes research. (Ayala, et al., 2011) We have adapted this technique for use in a SCID mouse xenograft model. FIG. 3 gives a schematic of this procedure. Briefly, mice are injected subcutaneously in the hind flank with a standard amount of cancer cells sufficient to cause a tumor to grow. After a few weeks when tumor volume measures around 300-500 mm3, mice undergo jugular vein cannulation. Mice are fasted for 5 hours prior to treatment with chemotherapy drugs. Depending on the pharmacokinetics of the drug, drugs are either given before the start of the glucose clamp or during the clamp procedure to correspond to the maximum effectiveness of the drug during the “hypoglycemia therapeutic window”. Injection pumps containing insulin, glucose and washed erythrocytes from donor mouse blood are connected to a swivel mixer which is in turn connected to the jugular catheters. For the initiation of the hypoglycemic clump, a bolus injection of insulin is given to reduce initial blood glucose levels which typically take around 30 minutes to lower blood glucose. Then a steady state of insulin and glucose is given to maintain blood glucose levels between 30-50 mg/dL for two hours. In practice, blood glucose levels range from 22-51 mg/dl with an average around 34 mg/dL (1.9 mmol/L). FIG. 4 shows results from two tests with different cancer cells and drugs. A549 cells are of human lung cancer origin and mice bearing A549 tumors were administered two treatments, 2 days apart, of pemetrexed, given I.V. before start of the clamp, 10 mg/kg, gemcitabine, given I.V. at the start of hypoglycemia, and cisplatin, given I.V. at the start of hypoglycemia, 0.5 mg/kg. HCT-116 cells are of colon cancer origin and mice bearing HCT-116 tumors were administered two treatments, 2 days apart, of 5-fluorouricil, given I.V. before start of clamp, 10 mg/kg, irinotecan, given I.V. at the start of hypoglycemia, 10 mg/kg, and cisplatin, given I.V. at the start of hypoglycemia, 0.5 mg/kg.

Warburg Effect Targeted Chemotherapy Delivery System

The apparatus is a machine which regulates blood glucose levels, monitors ECG rhythms and delivers chemotherapy drug treatment intravenously via a pump system. The apparatus is able to regulate blood glucose levels over a longer duration of time than a simple inject once method. While the apparatus lowers blood sugar levels to a hypoglycemic state, chemotherapy is delivered to the patient though several pumps located inside the apparatus. The chemotherapy drug is contained in proprietary cartridges designed to fit in only the chemotherapy pumps located inside the apparatus while insulin and glucose each have their own proprietary cartridges making improper loading of the machine impossible (i.e. the insulin cartridge will not physically fit into the pump for glucose or chemotherapy drug and vice versa). As this system is a direct pump feed, the apparatus also includes a magnetic mixer for diluting insulin, glucose and chemo drugs into saline (saline also delivered by pump in proprietary cartridge).

While the apparatus controls blood glucose levels and delivers chemotherapy drugs, it also has many safety features. The main safety concern with hypoglycemia is abnormal heart rhythms. While long duration (about 2 hours) of hypoglycemia with the use of a glucose clamp is generally safe in healthy individuals, we are treating cancer patients with various co-morbidities and extra care is needed. An integrated ECG monitor is included which is able to trigger the glucose pump to elevate blood sugar levels in the case of an irregular heartbeat and/or reduce or cease administration of insulin. An external manually controlled glucose syringe is also available which can be operated by medical personnel. For proper medication delivery, the apparatus includes a bar code reader (or RFID chip or other identifying mechanism) which properly identifies chemotherapy drug cartridges so medical personnel can correctly load the machine with the drug the patient is to receive and lights a LED over the correct pump camber for the corresponding drug. Further, the apparatus can use the identifying mechanism to confirm that cartridges are not being reused or are counterfeit by connecting to a central database via a wired or wireless connection and verifying the identified cartridge is valid and/or hasn't been used before.

The apparatus has integrated software that receives instrument feeds from the continuous blood glucose monitor and the ECG monitor and interprets those feeds for display and for pump actions such as addition of glucose due to blood glucose levels dropping below the target range. The software package can be preprogrammed to deliver precise doses of drug by controlling the pump piston movement and through the bar code reader can insure the proper chemotherapy drug is loaded as per the preprogrammed treatment protocol. The apparatus can suggest a protocol based on cancer type and other variables and/or accept a protocol via wired or wireless connection. If an accepted protocol varies from a suggested protocol, the apparatus can issue a warning, which may be overridden by medical personnel.

Chemotherapy Treatment Under Hypoglycemia

The apparatus lowers blood glucose levels with insulin and then administers a reduced level of chemotherapy drugs. These treatments are repeated frequently, several times a week, to achieve a clinical response. Long duration of hypoglycemia, up to 2 hours, during chemotherapy treatment is necessary as the half-life activity of many chemotherapy drugs are in this range. A standard treatment would be first to lower blood glucose levels by half of normal levels by injecting insulin intravenously. Then, while hypoglycemia is induced, chemotherapy drugs are delivered over a period of time, in minutes up to several hours. Hypoglycemia is maintained during the treatment time by injecting additional insulin or glucose to regulate blood sugar levels. ECG heart rhythms are monitored during this time to prevent any adverse cardiac events by injection of glucose to bring blood glucose levels back to normal in the case of an adverse cardiac event.

### Mechanical Features of the WETC Delivery System:

Continuous blood glucose monitoring system able to regularly relay (potentially up to the minute or real time) blood glucose levels.
Insulin/glucose pump regulatory system able to maintain predetermined blood glucose levels.
Multiple pump system able to deliver not only insulin and glucose but also several chemotherapy drugs.
Internal magnetic mixer which can mix insulin, glucose and/or chemotherapy drugs with saline to deliver a constant fluid flow into the patient even when the protocol calls for a slower flow rate of drug. The mixer is able to dilute out chemotherapy drugs into saline which may otherwise be at higher than desired concentration to deliver intravenously.
Uses wired or wireless technology to interface with blood glucose and/or ECG monitor.
Contains USB, firewire, wired Ethernet, wireless Ethernet, serial port and other computer interfacing ports to update firmware and download and upload patient data and verify cartridges.

### Safety Features of the WETC Delivery System:

ECG monitoring system able to alert the system to any adverse cardiac events and return patient to normal blood glucose levels.
A secondary calibration blood glucose monitor is integrated into the machine which tests fresh patient blood from lancets, intravenous (i.v.) or port draws to insure the continuous blood glucose monitor is properly reading blood glucose levels.
External glucose delivery syringe for manual glucose delivery.
Bar code reader and LED light system to insure correct drug is placed in the correct pump slot.
Unique fitting shaped cartridges which only fit into the correct pump chamber for insulin, glucose and drugs pumps.
Large display screen for easy to see read outs of current blood glucose levels and ECG status even across the room.
A pressure sensor will indicate a clogged port or i.v. line and turn off the machine pumps to prevent over pressure of the lines and veins.
Anti-reflux valve is placed in line with the main input line tube and inhibits back flow from the patient into the machine.
An in-line air detector prevents air embolisms from occurring by detecting and removing air from the main line into a waste container.
A system heater controls condensation build up which can occur if cold cartridges of insulin, glucose or drugs (typically stored cold) are placed into the device before acclamation to room temperature.

### Software Features of the WETC Delivery System

Software is programmable with patient information and their treatment protocol.
Software is able to store patient information and treatments given in local and remote databases.
Pump loading protocol interprets bar code labels on drug cartridges and lights an LED light so medical personnel correctly loads different pumps with the correct drug cartridge.
Software controls a display panel and speaker for visual and audio output.
Software reads monitor feeds from blood glucose and ECG monitors and interprets those feeds to pump out insulin or glucose to maintain proper blood sugar level.
Software is able handle adverse events such as irregular heartbeat and take appropriate action such as glucose injection and alerting medical personnel.
Software is able to control pumps to deliver a precise dose of chemotherapy drug over a variable amount to time by controlling the flow rate of the pump and overall volume delivered.
Software controls saline pump flow rate to send both drugs and saline to the mixer for proper drug dilutions.
Software has protocol for flushing/cleaning the system and clearing air from tubing.
Human interface with push buttons or touch pad display technology.
Uses password, swipe card and/or finger print recognition so only medical personnel can access machine functions.

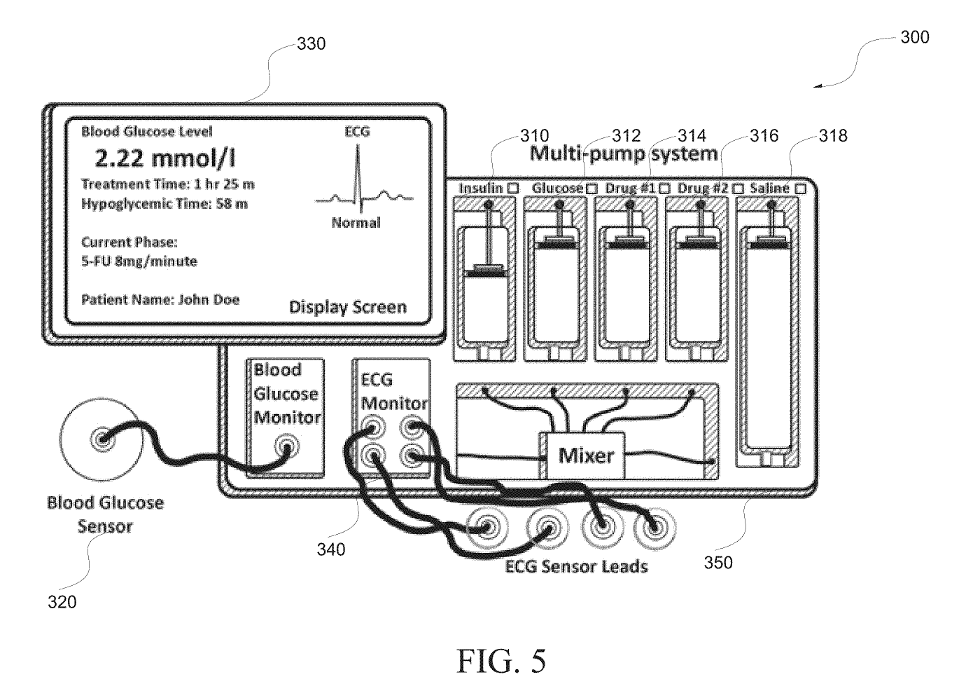

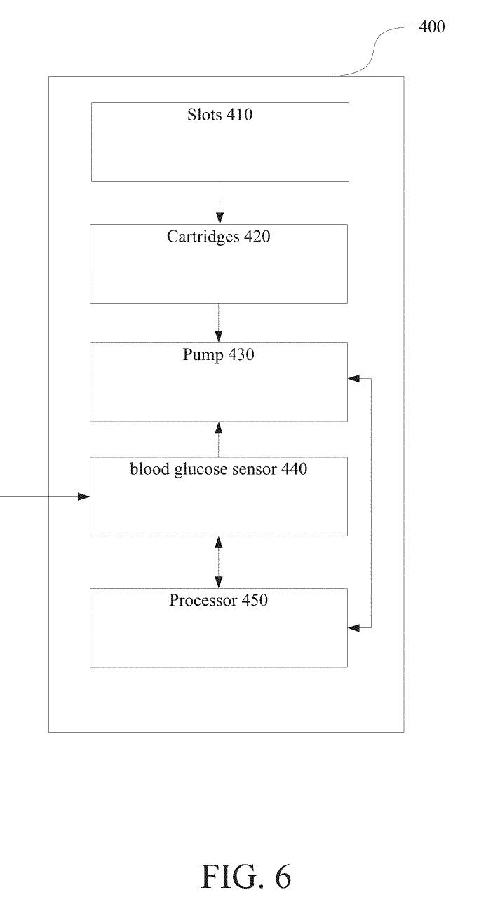

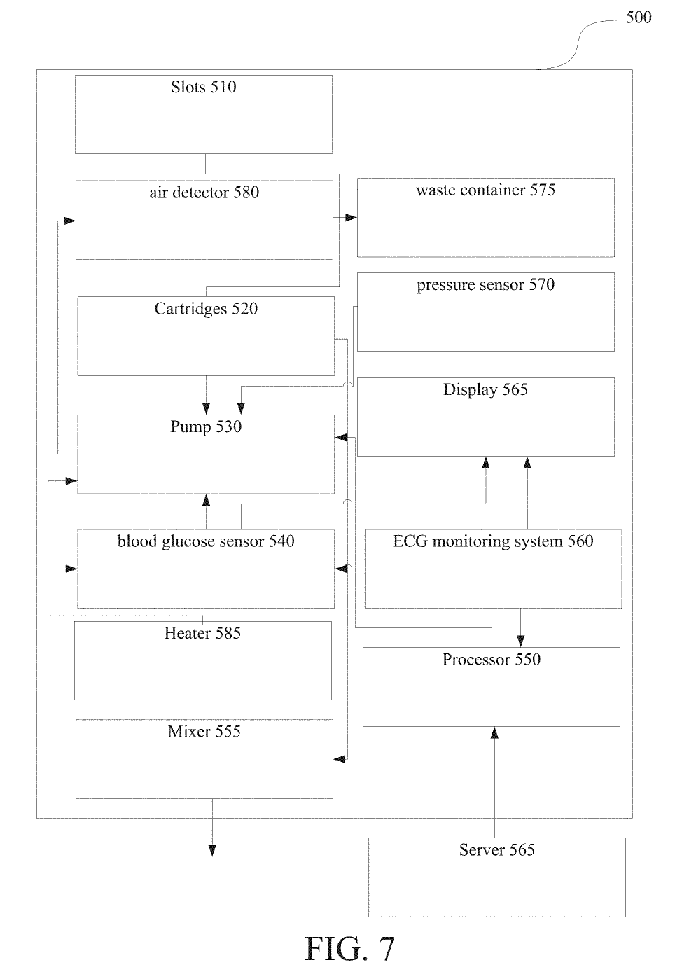

FIG. 5 is diagram for a system 300 for chemotherapy delivery according to an embodiment of the invention. The system 300 comprises a plurality of cartridges 310, 312, 314, 316 and 318 for insulin, glucose, chemotherapy drugs and/or other drugs, and/or saline, a plurality of pumps (not shown in FIG. 5) configured to receive the plurality of cartridges 310, 312, 314, 316 and 318, a blood glucose sensor 320, a mixer 350, a display 330 which can include a GUI, an ECG monitor system 340 with ECG leads, a processor (not shown in FIG. 5) that is communicatively coupled to the other components which will be discussed in further details with respect to FIG. 6 and FIG. 7.

FIG. 6 is a block diagram illustrating a system 400 for chemotherapy delivery according to an embodiment of the invention. Referring to FIG. 6, the system 400 for chemotherapy delivery comprises a plurality of slots 410, wherein each of the plurality of slots 410 is configured to receive a corresponding one of a plurality of cartridges 420; a plurality of pumps 430, wherein each of the plurality of pumps 430 is configured to be connected to the corresponding one of the plurality of cartridges, and the plurality of pumps 430 are configured to pump at least one drug contained in at least one of the plurality of cartridges 420 to a patient according to a treatment protocol. The at least one drug includes insulin, glucose and at least one chemotherapeutic drug, and the plurality of the cartridges 420 are configured to contain insulin, glucose and the at least one chemotherapeutic drug respectively. The system 400 further comprises a blood glucose sensor 440 communicatively coupled to the plurality of pumps 430, and configured to measure a blood glucose level of the patient; and a processor 450 connected to the plurality of pumps 430 and the blood glucose sensor 440 and configured to adjust delivery property of the at least one drug according to the measured blood glucose level of the patient; and wherein the plurality of pumps 430 are further configured by the processor 450 to adjust pumping the at least one drug according to the adjusted delivery property.

FIG. 7 is a block diagram for a system 500 for chemotherapy delivery according to another embodiment of the invention. The system 500 comprise a plurality of cartridges 520, a plurality of pumps 530, a blood glucose sensor 540, which are respectively similar to the plurality of cartridges 420, the plurality of pumps 430, the blood glucose sensor 440 shown in FIG. 6. Alternatively, one of the plurality of cartridges 420 contains saline, and the system 400 further comprises a mixer 555 connected to the plurality of cartridges 520 and the processor 550 and configured to dilute the at least one drug by diluting the insulin, the glucose and the at least one chemotherapeutic drug with the saline according to the adjusted delivery property, wherein the mixer 550 is further configured to deliver the diluted drug to the patient.

Alternatively, the system 500 further comprises an ECG monitoring system 560 communicatively coupled to the processor 550 and configured to monitor heart rhythm of the patient. The processor 550 is further configured to adjust the delivery property of the at least one drug according to the measured blood glucose level and the heart rhythm of the patient; wherein the plurality of pumps 530 are further configured by the processor 550 to adjust pumping the at least one drug according to the adjusted delivery property.

Alternatively, the ECG monitoring system 560 is further configured to indicate to the processor 550 that an adverse cardiac event is detected for the patient; and the processor 550 is further configured to instruct the plurality of pumps 530 to return the patient to normal blood glucose levels by changing the amount for pumping for insulin, and/or glucose.

Alternatively, the processor 550 is further communicatively connected to a server 565 and the processor 550 is further configured to download patient data from the sever 565; wherein the plurality of pumps 530 are further configured by the processor 550 to adjust pumping the at least one drug according to the patient data.

Alternatively, each of the plurality of slots 510 includes a chamber sized to receive the corresponding one of a plurality of cartridges.

Alternatively, the system 500 further comprises a display 565 communicatively coupled to both the ECG monitoring system 560 and the blood glucose sensor 540, and configured to show a current blood glucose levels and ECG status according to data received from the ECG monitoring system 560 and the blood glucose sensor 540.

Alternatively, the system 500 further comprises a pressure sensor 570 connected to the plurality of pumps 530 and configured to stop pumping of the plurality of pumps 530 if the pressure sensor 570 detects a blood pressure of the patient is higher than a threshold.

Alternatively, the system 500 further comprises a waste container 575, an air detector 580 connected to both the waste container 575 and an output line of the plurality of pumps 530 and configured to remove air from the output line into the waste container 575.

Alternatively, the system 500 further comprises a heater 585 placed in proximity to the plurality of cartridges 520 and configured to remove condensation in the at least one of the plurality of cartridges 520 by heating the at least one of the plurality of cartridges 520 to room temperature.

Alternatively, the delivery property of the at least one drug comprises the flow rate of drug delivery and volume of drug delivery, treatment time, treatment remaining, medication being administered, remaining medication to administer, medication administered, medication duration, order of drugs to be delivered.

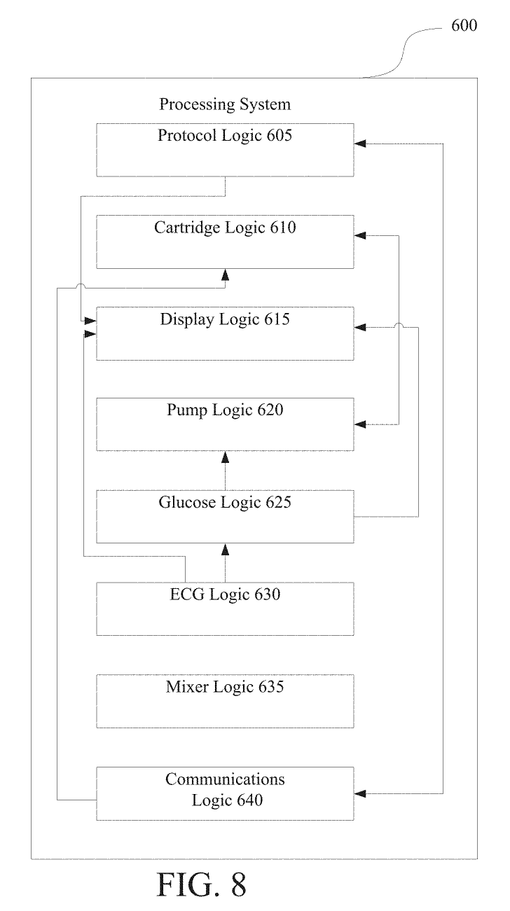

FIG. 8 is a block diagram illustrating the processing system 600. The processing system 600 includes protocol logic 605, cartridge logic 610, display logic (which can include GUI logic), pump logic 620, glucose logic 625, ECG logic 630, mixer logic 635, and communications logic 640.

During operation of the apparatus, the protocol logic 605 receives a protocol for a patient, either via manual entry (via an input device such as a GUI) or wired or wirelessly via the communications logic 640. In an embodiment, the protocol logic 605 can verify the protocol matches the type and/or stage of cancer. For example, breast cancer chemo should be administered for a breast cancer patient and not for a lung cancer patient. This verification can occur by checking a database within the apparatus and/or checking an external database in conjunction with the communications logic 640. In another embodiment, a user can enter the type of cancer and/or stage and receive a recommended protocol, which the user can then accept. Once the protocol is received, it is displayed by the display logic 615 on the display and in an embodiment, a user can accept the protocol (e.g., confirm it is correct for the correct person and affirmatively acknowledge it to prevent administering an incorrect medication to a patient).

Once protocol data is received/accepted, the cartridge logic 610 can indicate which cartridge goes into which pump by indicating the same via text and/or colors (e.g., cartridges and pumps can be color coded) adjacent the pumps and/or on the display. For example, the display could state insert an insulin cartridge in the leftmost pump and a display at the pump might read insulin and/or be color coded. Note that at least some of the pumps could have static labelling indicating the type of cartridge if that pump always using the same contents (e.g., the leftmost pump may always be used for insulin so an active display would not be needed).

In an embodiment, the above operation can be performed automatically without any human interaction. For example, upon the display states to insert an insulin cartridge in the leftmost pump, a robotic arm is programmed to insert an insulin cartridge in the leftmost pump according to predetermined instructions and the statement on the display.

Once a cartridge is inserted, the cartridge logic 610 further reads an identifying mechanism on each inserted cartridge to verify the correct cartridge is inserted in the respective pumps. Alternatively or in addition, the pump could be configured and/or shaped to accept only specific cartridges based on contents. The mechanism may be a bar code, RFID, magnetic strip, hologram, etc. In an embodiment, in conjunction with the communications logic 640, the cartridge logic 610 can contact a database to verify the authenticity of the cartridge and/or verify the cartridge is not being reused, which could lead to contamination problems and/or being refilled with counterfeit medication. For example, if a cartridge ID is not in the database, the cartridge is most likely counterfeit, meaning safety issues for the patient. If the cartridge ID is in the database but indicated as previously used, then cartridge could be counterfeit or refilled with potentially fake, contaminated, and/or unauthorized medications. If the verification fails, then the display logic 615 can present a warning re same and prevent administration of the cartridge contents. If verification passes, the database then updates itself to indicate the cartridge has been used.

In an embodiment, the cartridge logic 610 checks for an expiration date of the cartridge and will not enable the apparatus if a cartridge has expired.

In an embodiment, identification information that for example, the cartridge logic 610 uses to verify the authenticity of the cartridge and/or verify the cartridge is not being reused includes a checksum or other scheme to verify authenticity. This can be useful when it is not possible to connect with a database.

The display logic 615 displays messages and other information on the display. It receives the data to display from the other components of the apparatus, either directly or via the processing system. The display logic 615 can display ECG data, blood glucose data, administration instructions, progress information (e.g., phase information, hypoglycemic time, treatment time, treatment remaining, blood glucose level; medication being administered, remaining medication to administer, medication administered, etc.), patient data, protocol data, etc.

After the protocol has been received and the cartridge logic 610 verifies the cartridges, the pump logic 620 begins administration of the cartridge contents per the received protocol. During the administration, the protocol logic 605 updates the display with status of the administration. The administration, as discussed previously, includes administering of insulin to lower glucose blood levels and then administering the drugs. Note that the pump logic 620 may pump some or all of the cartridge content into the mixer before administering to the patient. For example, chemotherapy drugs and saline may be first pumped into the mixer before administering the mix to the patient. The glucose logic 625 receives blood glucose levels from the blood glucose monitor. The display logic 615 displays the blood glucose levels on the display. If the glucose logic 625 determines that blood glucose level is too low (e.g., 2.2 mmol/L), it will cause the pump logic 620 to administer glucose to the patient to increase the blood glucose level. Similarly, the ECG logic 630 receives data from the ECG monitor and if data is abnormal, will notify the glucose logic 625, which will in turn administer glucose as described above. Also note that in either case the glucose logic 640 or ECG logic 630 can cause the apparatus to issue a warning (audio, video, and/or text, etc.). In this case, a user can also manually administer glucose. Further, at completion of the protocol, the glucose logic 625 will cause the pump logic 620 to administer glucose to raise blood glucose levels to normal levels (e.g., >5.4 mmol/L).

The mixer logic 635 controls the mixer to mix the contents of the cartridges per the protocol. The mixer then administers the mix to the patient.

The communications logic 640 interacts via wired or wireless connection to receive a patient protocol and to interact with a database or databases per above.

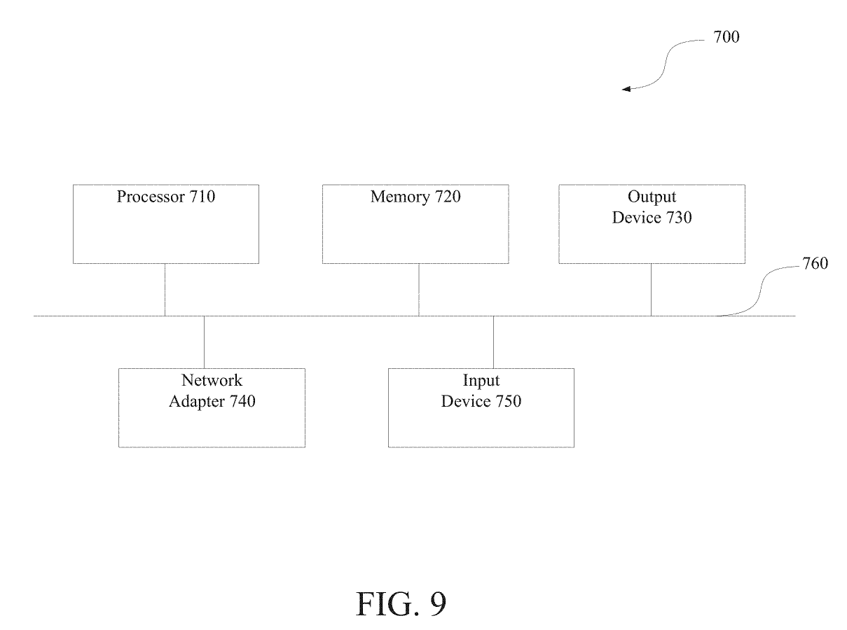

FIG. 9 is a high-level extent diagram showing an example of an architecture 700 of the system 300 of FIG. 5. The architecture 700 includes one or more processors 710 and memory 720 coupled to an interconnect 760. The interconnect 760 shown in FIG. 9 is an abstraction that represents any one or more separate physical buses, point to point connections, or both, connected by appropriate bridges, adapters, or controllers. The interconnect 760, therefore, may include, for example, a system bus, a form of Peripheral Component Interconnect (PCI) bus, a HyperTransport or industry standard architecture (ISA) bus, a small computer system interface (SCSI) bus, a universal serial bus (USB), IIC (I2C) bus, or an Institute of Electrical and Electronics Engineers (IEEE) standard 1394 bus, also called “Firewire”, and/or any other suitable form of physical connection.

The processor(s) 710 is/are the central processing unit (CPU) of the architecture 700 and, thus, control the overall operation of the architecture 700. In certain embodiments, the processor(s) 710 accomplish this by executing software or firmware stored in memory 720, which would therefore store the logics of FIG. 8, for example, protocol logics 605, cartridge logic 610, display logic 615, etc. The processor(s) 710 may be, or may include, one or more programmable general-purpose or special-purpose microprocessors, digital signal processors (DSPs), programmable controllers, application specific integrated circuits (ASICs), programmable logic devices (PLDs), or the like, or a combination of such devices.

The memory 720 is or includes the main memory of the architecture 700. The memory 720 represents any form of random access memory (RAM), read-only memory (ROM), flash memory, or the like, or a combination of such devices. In use, the memory 720 may contain, among other things, software or firmware code for use in implementing at least some of the embodiments of the invention introduced herein.

Also connected to the processor(s) 710 through the interconnect 760 is a communications interface, such as, but not limited to, a network adapter 740, one or more output device(s) 730 (e.g., the display of FIG. 5) and one or more input device(s) 750 (e.g., the display of FIG. 5 if touch sensitive). The network adapter 740 provides the architecture 700 with the ability to communicate with remote devices over the interconnect network 730 and may be, for example, an Ethernet adapter or Fiber Channel adapter. The input device 750 may include a touch screen, keyboard, and/or mouse, etc. The output device 730 may include a screen and/or speakers, etc.

The techniques introduced herein can be implemented by programmable circuitry programmed/configured by software and/or firmware, or entirely by special-purpose circuitry, or by a combination of such forms. Such special-purpose circuitry (if any) can be in the form of, for example, one or more application-specific integrated circuits (ASICs), programmable logic devices (PLDs), field-programmable gate arrays (FPGAs), etc.

Software or firmware to implement the techniques introduced here may be stored on a machine-readable storage medium and may be executed by one or more general-purpose or special-purpose programmable microprocessors. A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, etc.). For example, a machine-accessible medium includes recordable/non-recordable media (e.g., read-only memory (ROM); random access memory (RAM); magnetic disk storage media; optical storage media; flash memory devices; etc.), etc.

The term “logic”, as used herein, means: a) special-purpose hardwired circuitry, such as one or more application-specific integrated circuits (ASICs), programmable logic devices (PLDs), field programmable gate arrays (FPGAs), or other similar device(s); b) programmable circuitry programmed with software and/or firmware, such as one or more programmed general-purpose microprocessors, digital signal processors (DSPs) and/or microcontrollers, or other similar device(s); or c) a combination of the forms mentioned in a) and b).

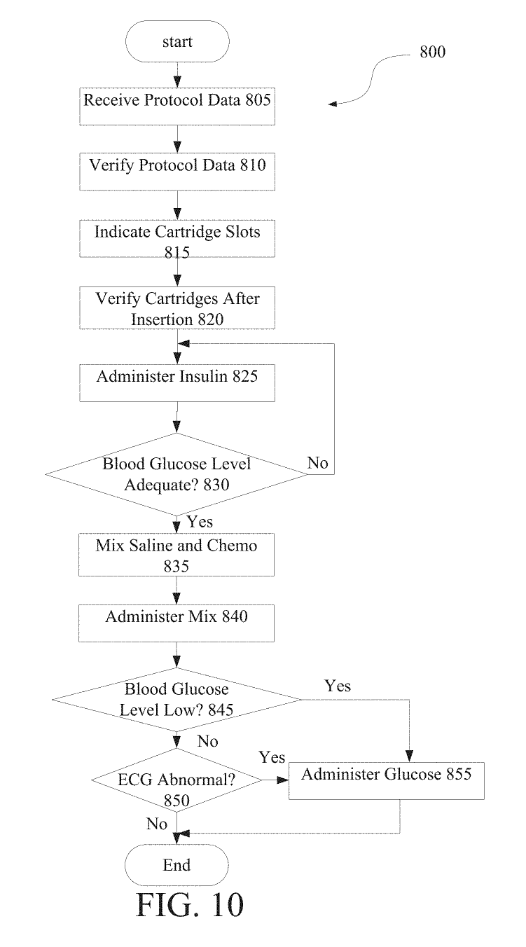

FIG. 10 is a flowchart illustrating of method of operation of the apparatus. In an embodiment, the logics of FIG. 8 can carry out the method. First, in block 805, protocol data is received. The protocol data includes, for example, medications (e.g., cancer therapy, insulin) and other agents (e.g., saline, glucose) to administer in order and duration and/or in combination. Examples protocols can be found in WO 2012/075679A1 attached hereto. In block 810, the protocol can then optionally be verified per above. In block 815, cartridge slots are then indicated which slots/pumps for which cartridges for each medication and agent, etc. In block 820, cartridges are then verified for authenticity, reuse, and/or expiration, etc. After verification, the method can include notifying a database to update cartridge records as used so as to prevent future refill and reuse.

In block 825, insulin is then administered to lower blood glucose level, which is monitored throughout the method. Insulin is continuously administered until a low blood glucose level is achieved (e.g., to 2.8-4.5 mmol/L or 2.2 mmol/L). When the method 800 determines that the blood glucose level is adequate, the method 800 proceeds with blocks 835 and 840, saline and medication (e.g., chemo) is mixed and the mix is administered. If the method 800 determines that the blood glucose level is not adequate, the method 800 goes back to block 825 to continue to administering insulin. Note that insulin can be continuously administered to maintain a target blood glucose level.

During the administration, as blood glucose levels are constant monitored, the method 800 determines if the blood glucose level is low in block 845. If the blood glucose falls below a predetermined level (e.g., 2.2 mmol/L), glucose is administered in block 855 to raise blood glucose levels. Further, during the method 800, if ECG is continuously monitored and if abnormal in block 850, glucose can be administered in block 855. Note that if the blood glucose falls too low and/or ECG is abnormal, the method can include performing a warning with sound and/or visually (e.g., on the display). The method then ends. Note that many portions of the method can be performed substantially simultaneously and/or in a different order than presented. Further, some portions can be omitted.

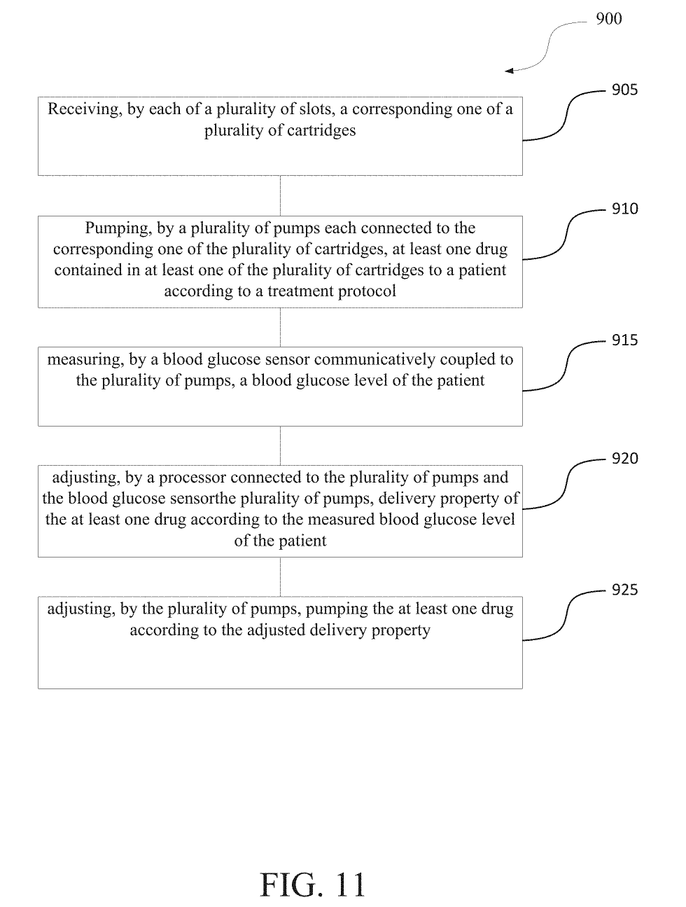

FIG. 11 is a flow diagram illustrating an example of method 900 of chemotherapy delivery according to an embodiment of the invention.

The method 900 comprises receiving, in block 905, by each of a plurality of slots, a corresponding one of a plurality of cartridges; pumping, in block 910, by a plurality of pumps each configured to be connected to the corresponding one of the plurality of cartridges, at least one drug contained in at least one of the plurality of cartridges to a patient according to a treatment protocol. The at least one drug includes insulin, glucose and at least one chemotherapeutic drug, and the plurality of the cartridges are configured to contain insulin, glucose and the at least one chemotherapeutic drug respectively. The method 900 further comprises measuring, in block 915, by a blood glucose sensor communicatively coupled to the plurality of pumps, a blood glucose level of the patient; adjusting, in block 920, by a processor connected to the plurality of pumps and the blood glucose sensor, delivery property of the at least one drug according to the measured blood glucose level of the patient; and adjusting, in block 925, by the plurality of pumps, pumping the at least one drug according to the adjusted delivery property.

Alternatively, the method 900 further comprises (not showing in FIG. 11) diluting, by a mixer connected to the plurality of cartridges and the processor, the at least one drug by diluting the insulin, the glucose and the at least one chemotherapeutic drug according to the adjusted delivery property, and delivering, by the mixer, the diluted drug to the patient.

Alternatively, the method 900 further comprises (not showing in FIG. 11) monitoring, by an ECG monitoring system communicatively coupled to the processor, heart rhythm of the patient; adjusting, by the processor, the delivery property of the at least one drug according to the measured blood glucose level and the heart rhythm of the patient; and adjusting, by the plurality of pumps, pumping the at least one drug according to the adjusted delivery property.

Alternatively, the method 900 further comprises (not showing in FIG. 11) indicating, by the ECG monitoring system, to the processor that an adverse cardiac event is detected for the patient; and instructing, by the processor to the plurality of pumps, to return the patient to normal blood glucose levels by changing the amount for pumping for insulin, and/or glucose.

Alternatively, the method 900 further comprises (not showing in FIG. 11) downloading, by the processor communicatively connected to a server, patient data from the sever; adjusting, by the plurality of pumps, pumping of the at least one drug according to the patient data.

Alternatively, each of the plurality of pumps includes a chamber sized to receive the corresponding one of a plurality of cartridges.

Alternatively, the method 900 further comprises (not showing in FIG. 11) showing, by a display communicatively coupled to both the ECG monitoring system and the blood glucose sensor, a current blood glucose levels and ECG status according to data received from the ECG monitoring system and the blood glucose sensor.

Alternatively, the method 900 further comprises (not showing in FIG. 11) stopping, by a pressure sensor connected to the plurality of pumps, pumping of the plurality of pumps if the pressure sensor detects a blood pressure of the patient is higher than a threshold.

Alternatively, the method 900 further comprises (not showing in FIG. 11) removing, by an air detector connected to both a waste container and an output line of the plurality of pumps, air from the output line into the waste container.

Alternatively, the method 900 further comprises (not showing in FIG. 11) removing, by a heater placed in proximity to the plurality of cartridges, condensation in the at least one of the plurality of cartridges by heating the at least one of the plurality of cartridges to room temperature.

Alternatively, the delivery property of the at least one drug comprises the flow rate of drug delivery and volume of drug delivery, treatment time, treatment remaining, medication being administered, remaining medication to administer, medication administered, medication duration, order of drugs to be delivered.

According to another embodiment, a computer readable storage medium, storing instructions when executed by a processor, cause the computer to perform operations comprising: controlling, a plurality of pumps each being configured to be connected to a corresponding one of a plurality of cartridges to pump at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively; controlling a blood glucose sensor communicatively coupled to the plurality of pumps to measure a blood glucose level of the patient; adjusting delivery property of the at least one drug according to the measured blood glucose level of the patient; and controlling the plurality of pumps to adjust pumping the at least one drug according to the adjusted delivery property.

Alternatively, one of the plurality of cartridges contains saline, and the operations further comprises controlling a mixer connected to the plurality of cartridges and the processor to dilute the at least one drug by diluting the insulin, the glucose and the at least one chemotherapeutic drug with the saline according to the adjusted delivery property, and controlling the mixer to deliver the diluted drug to the patient by adjusting a flow rate and volume of the delivered diluted drug.

Alternatively, the operations further comprises reading heart rhythm of the patient monitor monitored by an ECG monitoring system communicatively coupled to the processor; adjusting the delivery property of the at least one drug according to the measured blood glucose level and the heart rhythm of the patient; and controlling the plurality of pumps to adjust pumping the at least one drug according to the measured blood glucose level and the heart rhythm of the patient to maintain proper blood sugar level.

Alternatively, the operations further comprises monitoring a detection of an adverse cardiac event for the patient from the ECG monitoring system; and instructing the plurality of pumps to return patient to normal blood glucose levels by changing the amount for pumping for insulin, and/or glucose.

Alternatively, the operations further comprises downloading patient data from the sever; controlling the plurality of pumps to adjust pumping the at least one drug according to the patient data; and storing the adjusted delivery property in a data storage.

Alternatively, the operations further comprises controlling a display communicatively coupled to both the ECG monitoring system and the blood glucose sensor, to show a current blood glucose levels and ECG status according to data received from the ECG monitoring system and the blood glucose sensor.

Alternatively, the operations further comprises controlling an audio communicatively coupled to both the ECG monitoring system and the blood glucose sensor, to output an audio signal indicates a current blood glucose levels and ECG status according to data received from the ECG monitoring system and the blood glucose sensor.

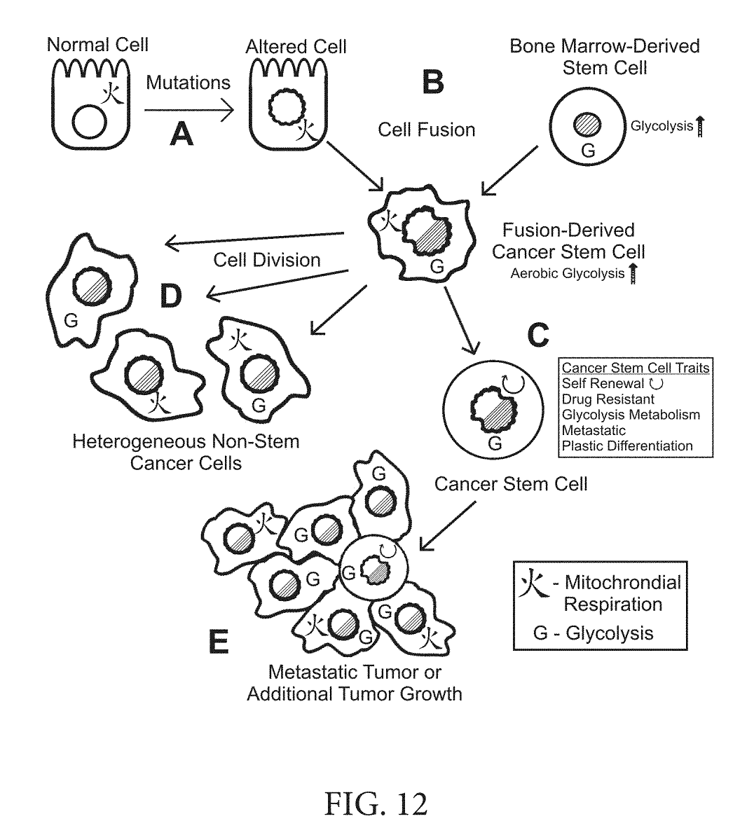

FIG. 12 is a schematic showing multi-step carcinogenesis of cancer stem cells (CSC) through cell fusion. A model for cancer stem cells been derived by fusion between genetically altered cells and bone-marrow-derived stem cells is shown by several steps. First, in step (A), genetic mutations lead to an altered hyperplasia cell phenotype leading to a benign neoplasm and local tissue damage. In step (B), bone marrow-derived stem cells, which use glycolysis as its metabolic energy source, are recruited to damage tissue and fuse with an altered cell. The progeny of the fusion will exhibit the hallmark of aneuploidy. In step (C), genetic mutations from the altered cells and epigenetic traits from the bone marrow-derived stem cells are combined to form a cancer stem cell. Stem cell traits include: self renewal, drug resistance, plasticity, glycolysis based metabolism (i.e., the Warburg effect), and capability to move about the body and forming metastasis. In step (D), Stem cell-like plasticity enables the cancer stem cell to divide and differentiate into a heterogeneous cancer cell population with different metabolic phenotypes. In step E, cancer stem cells are embedded within a primary and/or metastatic tumor. They have the ability for self renewal. Cancer stem cell survival from therapies allows regrowth and reoccurrence of cancer.

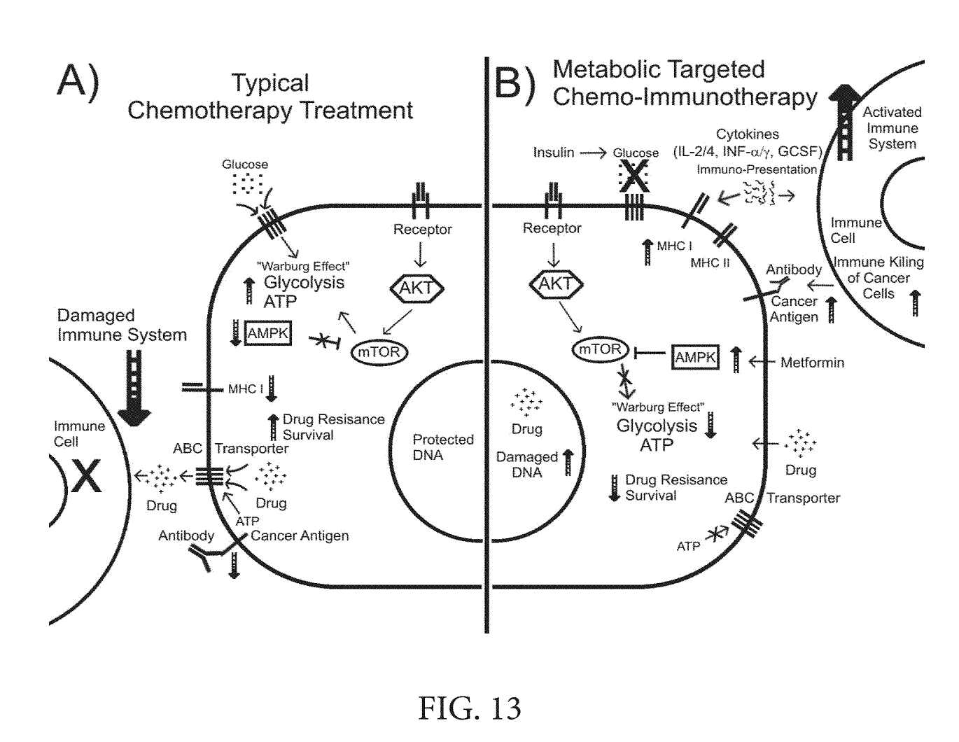

FIG. 13 is a schematic diagram illustrating comparisons of typical chemotherapy treatment versus metabolic targeted chemo-immunotherapy on cancer stem cells, according to the embodiments described herein. Cancer stem cells have a glycolysis based metabolism (i.e., the Warburg effect). Panel A shows that current chemotherapy protocols are administered without modifying the metabolism of the cancer stem cells. Chemotherapy given under typical conditions has glucose present for ATP production through glycolysis. ATP is consumed by ABC transporters which protect the cancer stem cells by exporting drugs. At the same time, standard high dose chemotherapy damages the immune system causing diminished effectiveness of antibody dependent cell toxicity and other immunotherapies, some of which utilize the major histocompatability complex I (MHC I) for recognition of tumor cells. Panel B shows that a combined therapy of metabolic targeting, immunotherapy and chemotherapy kills cancer stem cells. Insulin lowers blood sugar levels and depriving cancer stem cells of necessary glucose for ATP generation through glycolysis. In addition, Metformin disrupts signaling through the AKT pathway to further limit ATP production via glycolysis. ABC transporters starved of ATP are unable to pump drugs out of the cell leading to DNA damage. Lower, yet more frequent, doses of chemotherapy spares the immune system allowing antibodies and cytokines to enhance immune killing of cancer cells. Cytokine treatment of cancer stem cells activates the expression of MHC II which in turn presents tumor antigens to immune cells promoting additional immune responses against cancer cells.

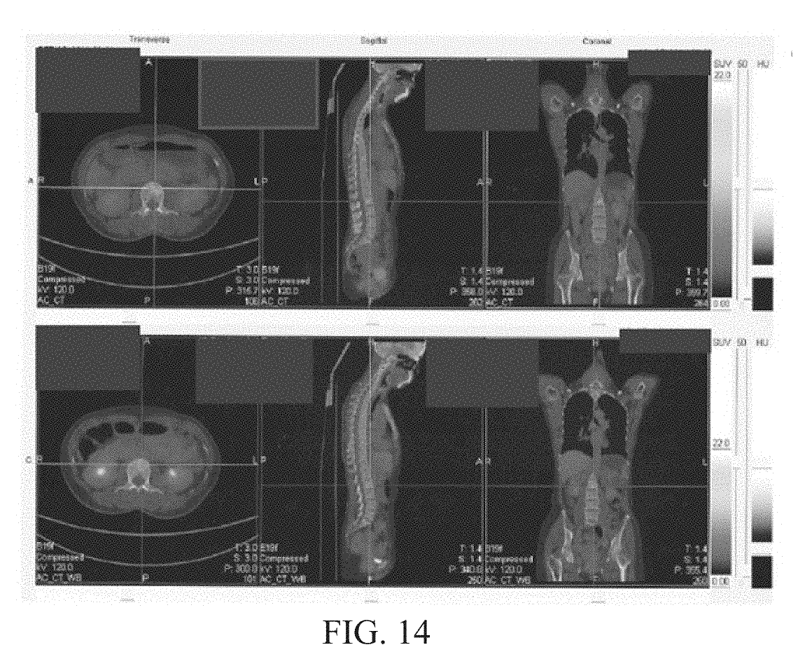

FIG. 14 shows representative PET/CT scans for an exemplar patient (Patient 4) receiving the metabolic targeting chemo-immunotherapy described in the embodiments herein. Said patient had extensive disease involvement in the L2 vertebrae with high PET SUV in the before treatment PET/CT scan (bottom panel). A follow up PET/CT scan post treatment showed nearly full regression of L2 vertebrae and bone regeneration (top panel).

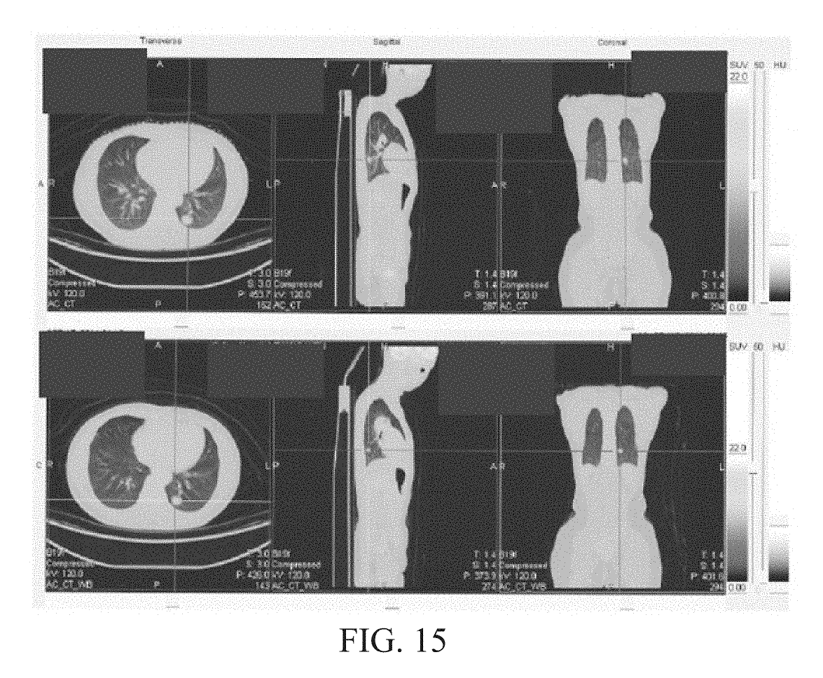

FIG. 15 shows a lung lesion from Patient 4. The mass measured 1.3×1.5×0.8 cm in the pre-treatment PET/CT scan (FIG. 15, bottom panel). SUV value was low, 1.5, indicating low glucose uptake in the lesion. A follow up PET/CT scan showed an increase of the left lung mass size, 1.3×1.5×1.8 cm, however, a slight decrease in SUV, 1.3, was also observed (FIG. 15, top panel).

Certain embodiments of the invention are described in detail, using specific examples, sequences, and drawings. The enumerated embodiments are not intended to limit the invention to those embodiments, as the invention is intended to cover all alternatives, modifications, and equivalents, which may be included within the scope of the present invention as defined by the claims. One skilled in the art will recognize many methods and materials similar or equivalent to those described herein, which could be used in the practice of the present invention.

An “agent,” “drug” or “therapeutic agent” refers to a chemical compound, a mixture of chemical compounds, a biological macromolecule, or an extract made from biological materials such as bacteria, plants, fungi, or 60 animal (particularly mammalian) cells or tissues that are suspected of having therapeutic properties. The agent or drug may be purified, substantially purified or partially purified. Examples of agents may include, but are not limited to, chemotherapeutic agents, targeted cancer therapies (e.g., therapeutic antibodies or functional fragments thereof, and immunologic agents, therapeutic antibodies. An “agent”, according to the present invention, also includes a radiation therapy agent.

“Antibody or functional fragment thereof” means an immunoglobulin molecule that specifically binds to, or is immunologically reactive with a particular antigen or epitope, and includes both polyclonal and monoclonal antibodies. The term antibody includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term functional antibody fragment includes antigen binding fragments of antibodies, including e.g., Fab′, F(ab′)2, Fab, Fv, rIgG, and scFv fragments. The term scFv refers to a single chain Fv antibody in which the variable domains of the heavy chain and of the light chain of a traditional two chain antibody have been joined to form one chain.

A “chemotherapeutic agent” is any agent used to treat cancer. Chemotherapeutic agents have many mechanisms of action, some of which are non-specific, affecting all cells in the body, while others are specific or targeted to cancer cells. The term chemotherapeutic agent includes all antineoplastic drugs, including small molecules, biologics, immunologic agents, targeted therapies, cytotoxic or cytolytic agents, alkylating agents, antimetabolites, anti-tumor antibiotics, topoisomerase inhibitors, or any other agent that is used to kill cancer cells, slow or stop cancer cell division, slow or stop cancer cell metastasis or otherwise treat cancer.

An “immunologic agent,” “immunotherapeutic” or “immunotherapeutic agent” is a substance or treatment having active or passive immunostimulant activity. Such activity may be a result of specific immunostimulants or non-specific immunostimulants. In addition to the above definition, the term “immunotherapy” includes behaviors or treatments that may directly or indirectly cause an increase in an immune response or causes an increase in an immune response relative to or in comparison to other therapies.

The term “immunostimulant” encompasses all substances, treatments or behaviors which influence the function of cells which are involved directly or indirectly in mediation of the immune response, and where the influence leads to an immune response. These cells include, for example, macrophages, natural killer cells, Langerhans cells and other dendritic cells, lymphocytes, indeterminate cells, fibroblasts, keratinocytes and melanocytes.

“In combination” or “in combination with,” as used herein, means in the course of treating the same disease in the same patient using two or more agents, drugs, treatment regimens, treatment modalities or a combination thereof, in any order. This includes simultaneous administration, as well as in a temporally spaced order of up to several days apart. Such combination treatment may also include more than a single administration of any one or more of the agents, drugs, treatment regimens or treatment modalities. Further, the administration of the two or more agents, drugs, treatment regimens, treatment modalities or a combination thereof may be by the same or different routes of administration. In the embodiments described herein, one or more chemotherapeutic agents may be administered, alone or in combination, to a subject for curative or palliative treatment of cancer.

A “pharmaceutically acceptable carrier” refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

“Route of administration” may refer to any administration pathway known in the art, including but not limited to aerosol, enteral, nasal, ophthalmic, oral, parenteral, rectal, transdermal (e.g., topical cream or ointment, patch), or vaginal. “Parenteral” refers to a route of administration that is generally associated with injection, including infraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal.

“Targeted cancer therapies,” “molecularly targeted drugs,” or “molecular targeted therapies” are drugs or other substances that block the growth and spread of cancer by interfering with specific molecular targets that are involved in tumor growth and progression. Although targeted cancer therapeutics may be considered as a type of chemotherapy, they are often considered a separate group. Targeted cancer therapies are typically a small molecule drug or a therapeutic antibody or functional fragment thereof.

“Treating” or “treatment” of a condition such as cancer may refer to preventing the condition, slowing the onset or rate of development of the condition, reducing the risk of developing the condition, preventing or delaying the development of symptoms associated with the condition, reducing or ending symptoms associated with the condition, generating a complete or partial regression of the condition, or any combination thereof.

A “standard dose” of a particular cancer treatment, including chemotherapeutics and targeted cancer therapies, is typically a maximum safe dosage. A “maximum safe dosage” or “maximum recommended therapeutic dosage” is the highest amount of a therapeutic agent that can be given that minimizes complications or side effects to a patient while maintaining its efficacy as a treatment. Such a dose can be adjusted to consider the patient's overall heath and any extenuating factors that could hamper the patient's recovery. Due to the severity and potential lethal outcome of the disease, a maximum safe dosage tolerated in cancer treatment may be an amount that causes considerable and severe side effects.

A “therapeutically effective amount,” “effective amount” or “effective dose” is an amount of a therapeutic agent that produces a desired therapeutic effect in a subject, such as preventing or treating a target condition or alleviating symptoms associated with the condition. The precise therapeutically effective amount is an amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, namely by monitoring a subject's response to administration of a compound and adjusting the dosage accordingly. For additional guidance, see Remington: The Science and Practice of Pharmacy 21st Edition, Univ. of Sciences in Philadelphia (USIP), Lippincott Williams & Wilkins, Philadelphia, Pa., 2005

In some embodiments, a therapeutically effective dose of a particular agent may be the same as or lower than a standard dose. In a preferred embodiment, the therapeutically effective dose is lower than a standard dose. A therapeutically effective dose for a particular agent used in accordance with the embodiments described herein may be a dose that is a fraction or a percentage of a standard dose for that particular agent. In some aspects, a therapeutically effective dose may be between about 1% and 99% of a standard dose, between about 1% and 90% of a standard dose, between about 1% and 80% of a standard dose, between about 1% and 70% of a standard dose, between about 1% and 60% of a standard dose, between about 1% and 50% of a standard dose, between about 1% and 40% of a standard dose, between about 1% and 30% of a standard dose, between about 1% and 20% of a standard dose, between about 5% and 20% of a standard dose, between about 1% and 10% of a standard dose or below about 10% of a standard dose for a particular agent. In one aspect, a therapeutically effective dose of a particular agent may be about 10% of a standard dose, about 1% of a standard dose, or lower than 1% of a standard dose for a particular agent. In yet another aspect, a therapeutically effective dose may be between about 0.1% and 1% of a standard dose, between about between about 0.01% and 1% of a standard dose or between about 0.001% and 1% of a standard dose for a particular agent.

Methods of treating cancer with an immunologic agent, one or more additional chemotherapeutic agents, lowering blood glucose or a combination thereof are provided. In some embodiments, methods for treating cancer include an immunologic agent, in combination with lowering blood glucose and administering one or more chemotherapeutic agents. Such embodiments may be part of a cancer treatment regimen known as a targeting chemotherapy regimen or a targeting chemo-immunotherapy regimen.

A method of metabolic targeting chemo-immunotherapy may include a metabolic targeting chemotherapy treatment regimen used in combination with an immunologic targeting treatment regimen, both of which are described further below. In one embodiment, the method includes administering a therapeutic antibody or functional fragment thereof in combination with administration of one or more chemotherapeutic agents under low blood glucose conditions.

The methods described herein may be used to treat any cancer or tumor type. Cancers and tumor types that may be treated using the methods described herein include but are not limited to bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer. In addition, the methods may be used to treat tumors that are malignant (e.g., cancers) or benign (e.g., hyperplasia, cyst, pseudocyst, hamartoma, and benign neoplasm).

Chemotherapy and Other Anti-Cancer Agents

Cancer treatment often involves chemotherapy alone or in combination with other modalities of treatments such as surgery, radiation therapy, targeted therapy and immunotherapy. Chemotherapy may be used to mean the use of any drug to treat any disease, but is often associated with cancer treatment. In the treatment of cancer, chemotherapeutics or chemotherapeutic agents are often referred to antineoplastic or anticancer agents. Many chemotherapeutic agents are cytotoxic or cytostatic in nature. Antineoplastic chemotherapeutic agents can be divided into several groups based on factors such as how they work, their chemical structure or source, and their relationship to another drug. Such groups include, but are not limited to, alkylating agents, antimetabolites, anti-tumor antibiotics, topoisomerase inhibitors, mitotic inhibitors hormone therapy, targeted therapeutics and immunotherapeutics. Some chemotherapeutics do not fit well into any categories. Because some drugs act in more than one way, they may belong to more than one group. Knowing how the drug works is important in predicting side effects.

Alkylating agents directly damage DNA to prevent the cancer cell from reproducing. These agents are not phase-specific, but instead work in all phases of the cell cycle. Alkylating agents are used to treat many different cancers, including acute and chronic leukemia, lymphoma, Hodgkin disease, multiple myeloma, sarcoma, as well as cancers of the lung, breast, and ovary. Because these drugs damage DNA, they can cause long-term damage to the bone marrow. Alkylating agents that may be used according to the embodiments of the disclosure include, but are not limited to, nitrogen mustards (e.g., mechlorethamine (nitrogen mustard), chlorambucil, cyclophosphamide, ifosfamide and melphalan), nitrosoureas (e.g., streptozocin, carmustine (BCNU) and lomustine), alkyl sulfonates (e.g., busulfan), triazines (e.g., dacarbazine (DTIC) and temozolomide) and ethylenimines (e.g., thiotepa and altretamine (hexamethylmelamine)). In addition, the platinum drugs (cisplatin, carboplatin, and oxalaplatin) may be used according to the embodiments of the disclosure and are sometimes grouped with alkylating agents because they kill cells in a similar way.

Antimetabolites interfere with DNA and RNA growth by substituting for the normal building blocks of RNA and DNA. These agents damage cells during the S phase of the cell cycle. They are commonly used to treat leukemias, tumors of the breast, ovary, and the intestinal tract, as well as other cancers. Antimetabolites that may be used according to the embodiments of the disclosure include, but are not limited to, 5-fluorouracil (5-FU), capecitabine, 6-mercaptopurine (6-MP), methotrexate, gemcitabine, cytarabine, fludarabine and pemetrexed.

Anthracyclines are anti-tumor antibiotics that interfere with enzymes involved in DNA replication. These agents are not phase-specific. Thus, they are widely used for a variety of cancers. A major consideration when giving these drugs is that they can permanently damage the heart if given in high doses. For this reason, lifetime dose limits are often placed on these drugs. Anthracyclines that may be used according to the embodiments of the disclosure include, but are not limited to, daunorubicin, doxorubicin, epirubicin, and idarubicin. Other anti-tumor antibiotics include the drugs actinomycin-D, bleomycin, and mitomycin-C. In addition, mitoxantrone is another anti-tumor antibiotic that is similar to doxorubicin in many ways, including the potential for damaging the heart. This drug also acts as a topoisomerase II inhibitor (see below). Mitoxantrone is used to treat prostate cancer, breast cancer, lymphoma, and leukemia.

Topoisomerase inhibitors interfere with enzymes called topoisomerases, which help separate the strands of DNA so they can be copied. They are used to treat certain leukemias, as well as lung, ovarian, gastrointestinal, and other cancers. Topoisomerase inhibitors that may be used according to the embodiments of the disclosure include, but are not limited to, topoisomerase I inhibitors (e.g., topotecan and irinotecan (CPT-11) and topoisomerase II inhibitors (e.g., etoposide (VP-16), mitoxantrone and teniposide).

Mitotic inhibitors are often plant alkaloids and other compounds derived from natural products. They can stop mitosis or inhibit enzymes from making proteins needed for cell reproduction. These drugs generally work during the M phase of the cell cycle, but can damage cells in all phases. They are used to treat many different types of cancer including breast, lung, myelomas, lymphomas, and leukemias. Mitotic inhibitors that may be used according to the embodiments of the disclosure include, but are not limited to, taxanes (e.g., paclitaxel and docetaxel), epothilones (e.g., ixabepilone), vinca alkaloids (e.g., vinblastine, vincristine, and vinorelbine), and estramustine.

Some chemotherapy drugs do not fit well into any of the categories described above because they act in slightly different ways. Examples include, but are not limited to, L-asparaginase, which is an enzyme, and the proteosome inhibitor bortezomib.

Additional chemotherapeutics may include glycolysis inhibitors. As explained in more detail below, cancer cells are primarily glycolytic, relying heavily on the glycolysis pathway to generate ATP. The use of glycolysis inhibitors are thought to significantly reduce ATP generation, thereby preferentially killing cancer cells (Pelicano et al. 2006). However, glycolysis inhibitors can have toxic effects on healthy tissues, such as the brain, that also rely on glycolysis for energy. Thus, as with other types of chemotherapeutics, lowering blood glucose levels would allow glycolysis inhibitors to be used at lower doses to sensitize and more effectively target cancer cells. Glycolysis inhibitors target components of the glycolytic pathway such as hexokinase (HK), glugose-6-phosphate dehydrogenase (G6PG), transketolase-like enzyme 1 (TKTL1), glyceraldehyde-3-phosphate dehydrogenase (GAPDH) and pyruvate dehydrogenase kinase (PDK). In addition, glycolysis inhibitors may be non-metabolizable glucose analogs. Examples of glycolysis inhibitors that may be used in accordance with the embodiments described herein include, but are not limited to, α-chlorohydrin, 6-aminonicotinamide (6-AN), arsenic compounds, 3-BrOP, 3-bromopyruvate (3-PrPA), bromopyruvic acid, 2-deoxy-D-glucose (2-DG), dichloroacetic acid, dichloroacetates and related salts, genistein, glufosfamide, imatinib, lonidamine, mannoheptulose, ornidazole, oxalate, oxythiamine, SB-204990, and 5-thioglucose.

Targeted cancer therapies block the growth and spread of cancer by interfering with specific molecules involved in tumor growth and progression. Targeted therapies may have properties or characteristics of more than one category of chemotherapeutic agents, including cytotoxic agents, hormone therapy, biologic therapy and immunotherapy. For example, therapeutic antibodies are biologic agents that have chemotherapeutic and immunotherapeutic characteristics, as described further below. In addition, although targeted therapies often provide an efficient method for tailoring cancer treatment based on the type of cancer, and/or the unique set of molecular targets produced by a patient's tumor, they have several limitations including side effects (e.g., allergic reactions, chills, fatigue, fever, muscle aches and pains, nausea, diarrhea, skin rashes, heart failure, skin infections and bleeding) and the potential for developing resistance to these therapeutics. In many cases, once resistance occurs, alternative targeted therapies do not exist.

Examples of targeted therapies that may be used in accordance with any of the treatment regimens described herein include, but are not limited to, selective estrogen receptor modulators (SERMs) (e.g., tamoxifen, toremifene and fulvestrant), aromatase inhibitors (anastrozole, exemestane and letrozole, kinase inhibitors (imatinib mesulate, dasatinib, nilotinib, lapatinib, gefitinib, erlotinib, temsirolimus and everolimus, growth factor receptor inhibitors (e.g., Trastuzumab, cetuximab and panitumumab), regulators of gene expression (vorinostat, romidepsin, bexarotene, alitretinoin and tretinoin), apoptosis inducers (bortezomib and pralatrezate), angiogenesis inhibitors (bevacizumab, sorafenib, sunitinib and pazopanib), antibodies that triggers a specific immune response by binding a cell-surface protein on lymphocytes (rituximab, alemtuzumab and ofatumumab), antibodies or other molecules that deliver toxic molecules specifically to cancer cells (tositumomab, ibritumomab tiuxetan, denileukin diftitox), cancer vaccines and gene therapy.

In some embodiments, chemotherapeutic agents, used alone or in combination, that may be used to treat cancer according to the embodiments described herein may include, but are not limited to, 13-cis-Retinoic Acid, 2-Chlorodeoxyadenosine, 5-Azacitidine, 5-Fluorouracil, 6-Mercaptopurine, 6-Thioguanine, actinomycin-D, adriamycin, aldesleukin, alemtuzumab, alitretinoin, all-transretinoic acid, alpha interferon, altretamine, amethopterin, amifostine, anagrelide, anastrozole, arabinosylcytosine, arsenic trioxide, amsacrine, aminocamptothecin, aminoglutethimide, asparaginase, azacytidine, bacillus calmette-guerin (BCG), bendamustine, bevacizumab, bexarotene, bicalutamide, bortezomib, bleomycin, busulfan, calcium leucovorin, citrovorum factor, capecitabine, canertinib, carboplatin, carmustine, cetuximab, chlorambucil, cisplatin, cladribine, cortisone, cyclophosphamide, cytarabine, darbepoetin alfa, dasatinib, daunomycin, decitabine, denileukin diftitox, dexamethasone, dexasone, dexrazoxane, dactinomycin, daunorubicin, decarbazine, docetaxel, doxorubicin, doxifluridine, eniluracil, epirubicin, epoetin alfa, erlotinib, everolimus, exemestane, estramustine, etoposide, filgrastim, fluoxymesterone, fulvestrant, flavopiridol, floxuridine, fludarabine, fluorouracil, flutamide, gefitinib, gemcitabine, gemtuzumab ozogamicin, goserelin, granulocyte—colony stimulating factor, granulocyte macrophage-colony stimulating factor, hexamethylmelamine, hydrocortisone hydroxyurea, ibritumomab, interferon alpha, interleukin-2, interleukin-4, interleukin-11, isotretinoin, ixabepilone, idarubicin, imatinib mesylate, ifosfamide, irinotecan, lapatinib, lenalidomide, letrozole, leucovorin, leuprolide, liposomal Ara-C, lomustine, mechlorethamine, megestrol, melphalan, mercaptopurine, mesna, methotrexate, methylprednisolone, mitomycin C, mitotane, mitoxantrone, nelarabine, nilutamide, octreotide, oprelvekin, oxaliplatin, paclitaxel, pamidronate, pemetrexed, panitumumab, PEG Interferon, pegaspargase, pegfilgrastim, PEG-L-asparaginase, pentostatin, plicamycin, prednisolone, prednisone, procarbazine, raloxifene, rituximab, romiplostim, ralitrexed, sapacitabine, sargramostim, satraplatin, sorafenib, sunitinib, semustine, streptozocin, tamoxifen, tegafur, tegafur-uracil, temsirolimus, temozolamide, teniposide, thalidomide, thioguanine, thiotepa, topotecan, toremifene, tositumomab, trastuzumab, tretinoin, trimitrexate, alrubicin, vincristine, vinblastine, vindestine, vinorelbine, vorinostat, or zoledronic acid.

In other embodiments, the one or more chemotherapeutic agents used in the methods described herein may correspond to known chemotherapeutic regimens known in the art including, but not limited to, ABVD, AC, BEACOPP, BEP, CA (or AC), CAF, CAV, CBV, ChlVPP/EVA, CHOP (or COHP), R-CHOP, COP (or CVP), CMF, COPP, EC, ECF, EP, EPOCH, FEC, FL (also known as Mayo), FOLFOX, FOLFIRI, ICE, ICE-R, m-BACOD, MACOP-B, MOPP, PCV, ProMACE-MOPP, ProMACE-CytaBOM, R-FCM, Stanford V, Thal/Dex, TIP, VAC, VAD, VAPEC-B, and VIP. Further explanation of these chemotherapeutic regimens is found in Table 1 below.

TABLE 1
Known Chemotherapeutic Regimens.

| | | Example of uses, and |
|---|---|---|
| Regimen | Components | other notes |
| ABVD | Adriamycin (doxorubicin), bleomycin, | Hodgkin's lymphoma |
| | vinblastine, dacarbazine | |
| AC | Adriamycin (doxorubicin), cyclophosphamide | Breast cancer |
| BEACOPP | Bleomycin, etoposide, Adriamycin | Hodgkin's lymphoma |
| | (doxorubicin), cyclophosphamide, Oncovin | |
| | (vincristine), procarbazine, prednisone | |
| BEP | Bleomycin, etoposide, platinum agent | Testicular cancer, germ |
| | (cisplatin) | cell tumors |
| CA | Cyclophosphamide, Adriamycin (doxorubicin) | Breast cancer |
| | (same as AC) | |
| CAF | Cyclophosphamide, Adriamycin (doxorubicin), | Breast cancer |
| | fluorouracil (5-FU) | |
| CAV | Cyclophosphamide, Adriamycin (doxorubicin), | Lung cancer |
| | vincristine | |
| CBV | Cyclophosphamide, BCNU (carmustine), VP- | Lymphoma |
| | 16 (etoposide) | |
| ChlVPP/EVA | Chlorambucil, vincristine (Oncovin), | Hodgkin's lymphoma |
| | procarbazine, prednisone, etoposide, | |
| | vinblastine, Adriamycin (doxorubicin) | |
| CHOP or | Cyclophosphamide, hydroxydoxorubicin | Non-Hodgkin lymphoma |
| COHP | (doxorubicin), vincristine (Oncovin), | |
| | prednisone | |
| CHOP-R or | CHOP + rituximab | B cell non-Hodgkin |
| R-CHOP | | lymphoma |
| COP or CVP | Cyclophosphamide, Oncovin (vincristine), | Non-Hodgkin lymphoma |
| | prednisone | in patients with history of |
| | | cardiovascular disease |
| CMF | Cyclophosphamide, methotrexate, fluorouracil | Breast cancer |
| | (5-FU) | |
| COPP | Cyclophosphamide, Oncovin (vincristine), | Non-Hodgkin lymphoma |
| | procarbazine, prednisone | |
| EC | Epirubicin, cyclophosphamide | Breast cancer |
| ECF | Epirubicin, cisplatin, fluorouracil (5-FU) | Gastric cancer and |
| | | oesophageal cancer |
| EP | Etoposide, platinum agent (cisplatin) | Testicular cancer, germ |
| | | cell tumors |
| EPOCH | Etoposide, prednisone, Oncovin, | Lymphomas |
| | cyclophosphamide, and hydroxydaunorubicin | |
| FEC | Fluorouracil (5-FU), epirubicin, | Breast cancer |
| | cyclophosphamide | |
| FL (Also | Fluorouracil (5-FU), leucovorin (folinic acid) | Colorectal cancer |
| known as | | |
| Mayo) | | |
| FOLFOX | Fluorouracil (5-FU), leucovorin (folinic acid), | Colorectal cancer |
| | oxaliplatin | |
| FOLFIRI | Fluorouracil (5-FU), leucovorin (folinic acid), | Colorectal cancer |
| | irinotecan | |
| ICE | ifosfamide, carboplatin, etoposide (VP-16) | Aggressive lymphomas, |
| | | progressive neuroblastoma |
| ICE-R | ICE + rituximab | High-risk progressive or |
| | | recurrent lymphomas |
| m-BACOD | Methotrexate, bleomycin, Adriamycin | Non-Hodgkin lymphoma |
| | (doxorubicin), cyclophosphamide, Oncovin | |
| | (vincristine), dexamethasone | |
| MACOP-B | Methotrexate, leucovorin (folinic acid), | Non-Hodgkin lymphoma |
| | Adriamycin (doxorubicin), cyclophosphamide, | |
| | Oncovin (vincristine), prednisone, bleomycin | |
| MOPP | Mechlorethamine, Oncovin (vincristine), | Hodgkin's lymphoma |
| | procarbazine, prednisone | |
| PCV | Procarbazine, CCNU (lomustine), vincristine | Brain tumors |
| ProMACE- | Methotrexate, Adriamycin (doxorubicin), | Non-Hodgkin lymphoma |
| MOPP | cyclophosphamide, etoposide + MOPP | |
| ProMACE- | Prednisone, doxorubicin (adriamycin), | Non-Hodgkin lymphoma |
| CytaBOM | cyclophosphamide, etoposide, cytarabine, | |
| | bleomycin, Oncovin (vincristine), methotrexate, | |
| | leucovorin | |
| R-FCM | Rituximab, fludarabine, cyclophosphamide, | B cell non-Hodgkin |
| | mitoxantrone | lymphoma |
| Stanford V | Doxorubicin, mechlorethamine, bleomycin, | Hodgkin's lymphoma |
| | vinblastine, vincristine, etoposide, prednisone | |
| Thal/Dex | Thalidomide, dexamethasone | Multiple myeloma |
| TIP | Paclitaxel, ifosfamide, platinum agent cisplatin | Testicular cancer, germ |
| | | cell tumors in salvage |
| | | therapy |
| VAC | Vincristine, Actinomycin, Cyclophosphamide | Rhabdomyosarcoma |
| VAD | Vincristine, Adriamycin (doxorubicin), | Multiple myeloma |
| | dexamethasone | |
| VAPEC-B | Vincristine, Adriamycin (doxorubicin), | Hodgkin's lymphoma |
| | prednisone, etoposide, cyclophosphamide, | |
| | bleomycin | |
| VIP | Etoposide, ifosfamide, platinum agent cisplatin | Testicular cancer, germ |
| | | cell tumors |

Cell Fusion Leads to Carcinogenesis

The common dogma of how cancer forms is a series of genetic mutations which alters cells and leads to disease. Many studies have focused on chemical carcinogens and genetic instability resulting in mutations or aneuploidy of cancer-related genes. While these mutations are readily observed, they do not account for some traits of cancer, such as limitless replication/self renewal, differentiation into a heterogeneous population, and the ability to migrate through the body and survive in different tissue environments as in metastasis. These are the traits associated with what are described as cancer stem cells.

The discovery that many cancers arise from or contain stem cells that retain characteristics of normal stem cells (e.g., bone marrow derived stem cells, BMDSC) that allow them to survive for the lifespan of the individual. These characteristics include a low rate of cell division, active DNA repair and the expression of several transport proteins that protect cells against toxins, which makes the cancer stem cells relatively resistant to radiation and chemotherapy.

Cancer stem cells (CSCs) may arise from a normal stem cell that has undergone malignant transformation by accumulating genetic mutations or other abnormalities. Alternatively, cancer stem cells may arise from a stem cell fusion model of carcinogenesis that includes a fusion between a genetically altered cell and a stem cell of bone marrow origin. (He 2005). The stem cell fusion model of carcinogenesis is explained in detail in U.S. Patent Application Publication No. 20090016961, which is a national application of International Patent Application No. PCT/US06/033366, filed Aug. 25, 2006, which is hereby incorporated in its entirety as if fully set forth herein. Stem cell fusion may represent a missing step in the understanding of carcinogenesis.

This “stem cell fusion model of carcinogenesis” provides insights on new strategies to target CSCs by targeting the common traits of bone marrow derived stem cells have with CSCs, namely their glycolysis based metabolism (i.e., Warburg effect, described further below). One additional trait of CSCs is their metabolism of glucose for energy.

Alterations in the AKT pathway is one of the most commonly seen transformation events in cancer. Additionally, high rates of glucose consumption (glycolysis) of cancer cells, commonly known as the Warburg effect, is correlated with a metastatic phenotype and a typical trait of cancer. The AKT pathway can regulate glycolysis through mTOR. Various inputs, such as insulin like growth factor (IGF-1) or hypoxia inducible factors send a single through the AKT pathway which activates mTOR, which in turn upregulates glycolysis enzymes (Elstrom 2004).

Tissue based hypoxia, which is a condition of low oxygen supply and often caused by a lack of blood flow, is commonly seen in poorly vascularized tumors. Cells in a hypoxic state use glycolysis for energy while mitochondrial respiration, which requires oxygen, is inhibited. Bone marrow stem cells survive in a hypoxic niche within marrow and obtain energy through anaerobic glycolysis (Simsek 2010). This process is regulated by hypoxia inducible factors (HIFs). CSCs express some of these HIFs and can also reside in a hypoxic niche within the tumor microenvironment. These cells use glucose for energy and maintain stem cell markers while in their hypoxic niche. (Heddlestin 2010).

Use of glucose for energy by cancer cells is retained even in the presence of oxygen. This aerobic glycolic trait, or Warburg effect, of cancer is often thought to be induced by mutations, especially in the AKT/mTOR pathway. However, the CSCs use of glycolysis for energy can in part be a result of epigenetic changes brought about by fusion with bone marrow derived stem cells, which also use glycolysis for their energy needs (FIG. 12). The use of glycolysis maintains the stem cell phenotype which allows CSCs to self renew. This trait is inherited by the stem cell component of carcinogenic fusion. Thus, the metabolism of CSCs provide a unique target for therapy.

Evidence suggests that not only do cancer stem cells exist in solid tumors, but that they contribute to the invasive, malignant phenotype of these cancers. For example, a tumorigenic breast cancer stem cell population representing about 1% of the cells was found to be highly tumorigenic in mice, whereas the non-stem cells in the tumor were very poorly tumorigenic (Al-Hajj et al., 2003). Similarly, stem cells with a capacity to self-renew and undergo pluripotent differentiation have been isolated from human brain tumors and from lung tissue (Dean 2009).

Cancer Stem Cells and Drug Transporters

Cancer is composed of a heterogeneous mix of cell populations. Although just a small component of the cell population, the cancer stem cells, are known to be able to recapitulate the entire heterogeneous population. This is best demonstrated by cancer regrowth after tumor debulking therapies such as surgery, chemotherapy and radiation. Many reports have demonstrated that CSCs are more resistant to chemotherapy agents than their non-stem cell counterparts. This is, in part, due to their ability to efflux drugs from the cell through use of multidrug resistance pumps, described in detail below.

This ability of CSCs is used in their isolation using Hoechst 33342 dye, in which the CSC population is able to exclude this fluorescent dye allowing the non-fluorescent CSC population to be selected. Targeting the CSC population has been of great interest and is currently being investigated in several clinical trials. (Winquist 2009)

As discussed above, cancer stem cells retain characteristics of normal stem cells. One such characteristic is a high expression levels of specific ATP-dependent ABC drug transporter (or multi-drug resistance (MDR) pump) genes, including, but not limited to, ABCB1 (which encodes the P-glycoprotein transporter), ABCC1 (which encodes the MRP1 transporter), ABCC2 (which encodes the MRP2 transporter), ABCG2 (which encodes the breast cancer resistance protein, BCRP), ABCA2 (which encodes ABC2), and ABCB11 (which encodes the “sister of P-glycoprotein,” SPGP) (Leonard et al. 2003). These genes are members of the ATP-binding cassette (ABC) transporter superfamily and represent the major tumor multi-drug resistance genes. Expression of these MDR pumps allows cells to pump drugs out, conferring resistance to chemotherapeutics including, but not limited to, adramycin, daunorubicin, epirubicin, paclitaxel, docetaxel, vincristine, vinblastine, VP-16, mitoxantrone, actinomycin-D, doxorubicin, topoisomerase I or II inhibitors and anthracyclines (Leonard et al. 2003). Tumors that recur after an initial response to chemotherapy are often multi-drug resistant (Gottesman et al., 2002).

The drug transporting property of stem cells is an important phenotype for the isolation of hematopoietic stem cells. Stem cells exclude fluorescent dyes because the dyes are removed by ABCG2 and ABCB1. Therefore stem cells can be sorted by collecting the cells that contain only a low level of fluorescence referred to as the “side population” (SP cells, SP phenotype). Because stem cells are predominantly found in the SP fraction, it is possible to sort and purify stem cells from virtually any population of cells or tissue, including cancer. SP cells were identified in 15 out of 23 neuroblastoma samples and in neuroblastoma, breast cancer, lung cancer, and glioblastoma cell lines. Furthermore, analysis of several cell lines demonstrated a small population of SP cells. Therefore, even long-established tumor cell lines contain a cancer stem cell population, strongly supporting the idea that this is a fundamental property of cancers.

In a tumor stem cell paradigm, the cancer stem cells are naturally resistant to chemotherapy through their quiescence, their capacity for DNA repair, and ABC transporter expression. As a result, at least some of the tumor stem cells survive chemotherapy and support re-growth of the tumor. The resistance phenotype of the cancer stem cell persists in the committed, abnormally developing progenitors that comprise the recurrent tumor. Therefore, cancer stem cells may account for recurrence of cancer after treatment by surviving traditional cancer therapies (Dean 2009).

By inhibiting chemotherapy drug transporters, resistance may be overcome allowing complete elimination of the tumor. Therefore, ABC transporter inhibitors may be used to target cancer stem cells expressing drug transporters that make them resistant to many chemotherapy agents. ABCB1 inhibitors have shown limited effectiveness in clinical trials, however, these studies have not focused on targeting cancer stem cells (Dean 2009).

Metabolic Targeting Chemotherapy Treatment

Due to their dependence on glycolysis for energy, CSCs can be killed directly or indirectly by targeting glycolysis, either through antagonists of glycolysis metabolism and/or limiting the availability of glucose. Additionally, CSCs drug resistance can be overcome by limiting glycolysis through mechanisms which inhibit ATP production necessary to run ABC transporters, as previously described.

A negative regulator of the AKT/mTOR pathway is AMP-activated protein kinase (AMPK), which is activated in response to an increased ratio of AMP to ATP. An example of this function would be when low blood glucose levels limit ATP produced by glycolysis leading to an increase in AMP. The AMPK “energy sensor” directs increased consumption of fatty acids metabolism through mitochondria respiration and a decrease in glycolysis. In the context of CSCs, modulating this pathway to decrease glycolysis can deprive the CSCs of necessary ATP (Xu 2005).

Metformin is derived from French lilac, which has been used for centuries to treat symptoms of diabetes mellitus. Metformin belongs to a class of drugs called biguanides, which acts in an indirect manner on AMPK, which in turns suppresses the AKT/mTOR pathway. In the diabetic setting, metformin suppresses hepatic gluconeogenesis, the liver's ability to make glucose and thereby lower blood glucose levels.

People with diabetes have an increased risk of dying from cancer. However, patients taking metformin have a reduced cancer risk and a lower cancer related mortality. In an epidemiological study of 2,529 women with breast cancer, diabetic women taking metformin had a higher pathological complete response (pCR) rate to neoadjuvant systematic therapy compared to both diabetic and non-diabetic woman not taking metformin (diabetic metformin group—24% pCR, diabetic control—8% pCR, non-diabetic control—16%) (Dowling 2011; Hirsch 2009). Due to metformin's inhibition of the AKT/mTOR pathway via AMPK, glycolysis of CSCs can be impacted in a negative fashion leading to increased susceptibility of drugs and cell death.

Most chemotherapy agents act directly or indirectly on DNA. DNA is most vulnerable to damage while unwound from chromatin and histones to undergo DNA replication during S phase. Cancer cells are known for their uncontrolled cell growth. However, normal cells are growth restricted based on nutrient availability and can sense overall energy levels and postpone cell division. Under fasting conditions and/or hypoglycemia and/or modulation of metabolic pathways, normal cells stop the cell division process and go into a G1 cell cycle block.

Mutations in growth pathways common in cancer cells can inhibit this response. This leads to a “differential stress response” where normal cells are protected from the effects of chemotherapies while dividing cancer cells are left vulnerable (Raffaghello 2008). Normal cells and the tissues they make up are more resistant to chemotherapy under hypoglycemic or metabolically shifted conditions leading to fewer side effects and allowing for more frequent or higher dose treatments.

Therefore, in some embodiments, the one or more chemotherapeutic agents used in the methods described herein may include a plurality of chemotherapeutic agents, each of which target a different point in the cell cycle. This results in targeting a higher percentage of cancer cells or cancer stem cells with each dose.

Standard chemotherapy is generally administered once every 3 weeks. These drugs are often administered intravenously over several hours. Since cancer cells are their most vulnerable to many chemotherapeutic drugs during S phase, the frequency of chemotherapy treatments (i.e., how often it is administered) is important to the success of therapy.

Cancer cells are killed in a dose-dependant manner with increasing dose leading to more cell death. Dose dense regimens, or high frequency regimens, where chemotherapy is administered more often and at a higher overall dose, have been used in the past with mixed results but higher toxicity (Bonilla 2010). Continued administration of these treatments are inhibited by the higher toxicity and the efficacy is limited by the CSCs resistance to therapy.

The strategy for treatment with chemotherapy described herein greatly relieves many of the side effects experienced with standard therapy and as such, side effects are not a constraint to frequent therapy. Cancer cells undergo their vulnerable S phase frequently, but not frequent enough for most of them to be affected by standard chemotherapy schedules. Alternative mitotic blockers, a class of drugs which target microtubules, are only effective during M2 phase of cell division.

Different chemotherapeutic drugs targets different parts of the cell cycle. Because the time window during which the blood glucose level is lowered (minutes) is too short as compared to the duration of a cancer cell's cell cycle (hours or days), only a small fraction of cancer cells will be targeted and/or affected per treatment.

The cell cycle “window of opportunity” occurs for most of the cancer cell population outside of the pharmacological activity of the administered drugs. Even dose dense therapies are often only given weekly, leaving many cancer cells in a protected cell cycle phase during treatment. However, killing of cancer cells, and in particular, killing of cancer stem cells, may be enhanced by increasing the frequency of the treatments. To maximize the therapeutic efficacy, one should treat with a high frequency (i.e., as many times as possible) to target different parts of cell cycle. Thus, in some embodiments, the frequency of administering a chemotherapeutic agent is a high frequency and is selected from daily, every other day, 3 times weekly or biweekly.

In some embodiments, the more frequent or high frequency therapy is combined with conditions which not only sensitize cancer cells and CSCs to chemotherapy but also under conditions which protects normal cells. Delivering more frequent chemotherapy under hypoglycemic/metabolically modulated conditions will yield fewer side effects while increases efficacy by damaging cells in vulnerable cell cycles. Additionally, affecting glycolysis will increase sensitivity of CSCs to treatment by limiting ATP needed to run ABC transporters.

In some embodiments, a method for treating cancer may include a metabolic targeting chemotherapy treatment regimen. The metabolic targeting chemotherapy treatment regimen includes administering a therapeutically effective amount of one or more chemotherapeutic agents that is lower than a standard dose in combination with a method for lowering a subject's blood glucose to sensitize the cancer cells to the one or more chemotherapeutic agents. Sensitization of cancer cells to a chemotherapeutic agent in response to a decrease in blood sugar, renders the cancer cells more sensitive to the effects of the chemotherapeutic agent as compared to healthy cells (i.e., low blood sugar potentiates a chemotherapeutic agent's effect in cancer cells). Potentiation of a chemotherapeutic agent's effect results in an effective amount of chemotherapy to be lower than a standard dose, thereby reducing the side effects caused due to damage or death to healthy cells.

In other embodiments, a metabolic targeting chemotherapy treatment regimen for treating cancer may include a combination therapy, which affects multiple aspects of metabolism and provides synergistic efficacy not seen with individual compounds or treatments. The combination therapy includes administering an effective amount insulin to lower blood glucose and also administering an effective amount of metformin, both of which will inhibit glycolysis by limiting the supply of glucose and inhibiting pro-glycolysis signaling, respectively. Alternatively, the combination therapy includes administering an effective amount of metformin alone prior to treatment with a chemotherapeutic agent. In some embodiments, the administration of metformin may be continued after the administration of the chemotherapeutic agent is stopped or finished. Further, in one embodiment, the methods described herein may include administration of insulin or an insulin-dependent agent to induce hypoglycemia, followed by administration of metformin concurrently with one or more chemotherapeutic agents. The administration may be continued after the administration of the one or more chemotherapeutic agents has been stopped.

This is turn will reduce available ATP, necessary for the function of ABC transports which actively remove drug for CSCs. Additionally, limiting side effects of chemotherapy drugs by lowering blood glucose during treatment and using lower doses of the drugs allows for a treatment using immune system modulators to work more efficiently and allows for more frequent treatments.

In some embodiments, a metabolic targeting chemotherapy treatment regimen may include administering a therapeutically effective dose of one or more chemotherapeutic agents to a subject having cancer after lowering said subject's blood glucose level. The chemotherapeutic agent may be, but is not limited to, any of the agents or combination regimens described above or a combination thereof.

Lowering of a subject's blood glucose in accordance with the embodiments of the disclosure may be accomplished by one or more insulin dependent or insulin independent methods. Insulin independent methods that may be used include, but are not limited to, fasting a patient for a predetermined time, administering a low carbohydrate or low glycemic diet to a patient for a predetermined time and administering a drug that lowers blood glucose independent of insulin or the insulin receptor. Insulin dependent methods for lowering blood glucose include, but are not limited to, administering a dose of insulin to the patient or administering IGF-1 or any other suitable insulin receptor agonist to the patient.

In one embodiment, lowering of the subject's blood glucose level is accomplished by fasting a patient for a predetermined time. In one aspect, a patient may be fasted overnight to lower blood sugar to a baseline or fasting blood glucose level. A baseline blood glucose level is approximately 4.5-5.5 mmol/L. In another aspect, a patient may be fasted for a longer period of time to reduce blood glucose levels further. Such fasts may be used alone or may be supplemented by a diet having no sugar or carbohydrates or no sugar or carbohydrates. In another embodiment, lowering of the subject's blood glucose level is accomplished through administering a low glycemic diet comprising certain complex carbohydrates or a diet comprising no or low amounts of sugar or carbohydrates. Such diets may be used in combination with longer fasts.

In another embodiment, lowering of the subject's blood glucose level is accomplished by administering a drug that lowers blood glucose independent of insulin or the insulin receptor. Insulin independent drugs suitable for use with the embodiments described herein include, but are not limited to, (i) biguanides (e.g. metformin) that decrease the amount of glucose produced by the liver and alpha-glucosidase inhibitors (e.g., acarbose and meglitol) that block the breakdown of starches and sugars in the intestine; and (ii) sulfonylureas (e.g., glimepiride, glyburide and glipizide), which are not insulin or insulin agonists, however, they work in an insulin dependent manner by increasing the release of insulin by the pancreas. Taken alone or in combination with fasting they can cause drug induced hypoglycemia. Sulfonylureas are taken as individual compounds or in combination with metformin in a single pill. Examples of sulfonylureas are glimepiride, glyburide and glipizide. These insulin independent agents or drugs may be used in combination with administration of a chemotherapeutic in accordance with the embodiments of the disclosure. In some embodiments, one or more insulin-independent agents may be administered during the administration of one or more chemotherapeutic, after the administration of a chemotherapeutic, or a combination of both.

In another embodiment, lowering of the subject's blood glucose level is accomplished by administering a dose of insulin or a suitable insulin receptor agonist sufficient to reduce blood glucose levels. In one embodiment, the dose of insulin is approximately 0.1 to 0.5 units/kg. In another embodiment, the dose of insulin is approximately 0.2 units/kg. In one aspect, the dose of insulin is sufficient to reduce blood glucose level to roughly half of a baseline or fasting blood glucose level, to about 2.2-2.8 mmol/L. In another aspect, the dose of insulin is sufficient to reduce blood glucose level to about 2.8-4.5 mmol/L. In another aspect, a dose of insulin that is sufficient to reduce the blood glucose level to below 2.2 mmol/L may be used, however, is more dangerous to the patients.

Reducing blood sugar may potentiate the effect of chemotherapeutic agents through several mechanisms. Lowering blood glucose exploits the reliance on glucose by cancer cells to produce energy, also known as the Warburg effect (Warburg 1925). Cancer cells are glycolytic, and must consume glucose for their energy needs. The glucose is consumed anaerobically by cancer cells, yielding less energy than aerobic respiration. As a consequence, cancer cells must consume a large amount of glucose. Because of this effect, any method to lower blood glucose in a cancer patient may put cancer cells in a starvation state, making the cancer cells more sensitive to chemotherapeutic agents.

In addition to placing all cancer cells in a starvation state, lowering blood glucose levels in an insulin-independent manner to exploit the Warburg effect may also target cancer stem cells in particular, making them more vulnerable to chemotherapeutics. As discussed above, cancer stem cells retain many characteristics of normal stems cells, including chemoresistance characteristics as a result of increased expression of multi-drug resistance (MDR) pumps (Leonard et al. 2003). Because MDR pumps need ATP to work, lowering blood glucose levels reduces the available ATP available to the cancer stem cells to pump the chemotherapeutic drugs out of the cell, thereby disrupting the MDR pumps and overcoming the multi-drug resistance often seen in tumors. Further, targeting of MDR pumps in cancer stem cells by reducing blood glucose levels may be enhanced by administering a therapeutically effective dose of one or more MDR pump inhibitors in combination with a reduction in blood glucose.

Therefore, according to some embodiments, a metabolic targeting chemotherapy treatment may include administering a dose of one or more chemotherapeutic agents that is lower than a standard dose in combination with a method for disrupting ATP-dependant MDR pumps in cancer stem cells. The method for disrupting MDR pumps in cancer stem cells include reducing blood glucose levels in an insulin independent manner and/or administering one or more MDR pump inhibitors. Examples of MDR pump inhibitors suitable for use with embodiments of the disclosure include, but are not limited to, verapamil, quinidine, quinine, cyclosporine A, PSC 833, VX-710, LY335979, R101933, OC144-093 and XR9576 (Leonard et al. 2003).

In addition, the use of insulin, an insulin receptor agonist for lowering blood sugar may further potentiate the effect of chemotherapeutic agents in cancer cells. Cancer cells from many types of cancer have been observed to have more insulin receptors than normal cells (Ayre et al. 2000; Abita et al. 1984). Therefore, the use of insulin or an insulin receptor agonist to decrease blood glucose levels is thought to increase permeability of cancer cells to a greater extent than in normal cells, making the cancer cells more vulnerable to chemotherapeutic agents, increasing their efficacy. (Ayre et al., 2000). For example, a 2-fold increase in the uptake of elipticine by MDA-MB-231 breast cancer cells has been observed when the cells were incubated with insulin (Oster et al. 1981). Another study showed a 50% stimulation of uptake of methotrexate by breast cancer cells when the cells were incubated with insulin (Shilsky et al. 1971).

In some embodiments, the reduction in blood sugar is a maximized reduction in blood sugar. A maximized reduction in blood sugar is a reduction that corresponds to a lowest safe dosage the patient can tolerate. A maximized dosage may be as low as approximately 1.2 mmol.L or lower, depending on the patient's tolerance as assessed in the clinic.

Further, in some embodiments, the reduction in blood sugar is maintained for a prolonged period of time, i.e., the reduction is maintained for as long as safely practicable. The longer one can safely keep the blood sugar down, the more favorable result will occur.

This increase in cancer cell permeability and resulting increased uptake of chemotherapeutic agents may be due to an increase in insulin receptor or IGF-1 receptor-mediated transport (Poznansky et al. 1984; Yoshimasa et al. 1984; Gasparro et al. 1986; Ayre 1989), but may also be due to alterations in cellular lipid synthesis causing an increase in membrane fluidity (Jeffcoat & Jame 1984; Shinitzky et al. 1971; Jeffcoat 1979).

Insulin can also stimulate division of cancer cells, increasing the S-phase fraction in tumors, rendering the cells more susceptible to the cytotoxic effects of chemotherapeutic agents. Many chemotherapeutics act by targeting rapidly dividing cells as discussed above. Cancer cells are rapidly dividing cells, but only some cells are actively growing at any time, which means you can only kill some of the malignant cells at any time with conventional chemotherapy. Because insulin stimulates division in cancer cells, a higher percentage of the cancer cells divide at the same time, enabling chemotherapeutics to be absorbed by a much higher percentage of cancer cells. One study has shown that the addition of insulin to an asynchronous population of breast cancer cells increased the S-phase fraction to 66%, compared to only 37% in controls (Gross et al. 1984). Given the pharmacokinetics of neoplastic agents, particularly the cell-cycle phase specific agents, such an increase in the S-phase fraction would likely have a significant effect to enhance anticancer drug cytotoxicity.

In vitro data has shown the potentiation of chemotherapeutic agents in response to insulin. For example, when the chemotherapeutic agent methotrexate is administered with insulin to breast cancer cells in culture, the same percent cell killing is achieved with concentrations of methotrexate that are 104 lower than when methotrexate is administered alone (Alabaster et al. 1981).

Another receptor often found in greater numbers in cancer cells than in normal cells of the same tissue type is the insulin-type growth factor-1 receptor (IGF-1 receptor or IGF-1R). IGF-1 is a peptide of 70 amino acid residues having 40% identity with proinsulin. Insulin and IGF-1 have some cross-reactivity with each other's receptor. Therefore, in some embodiments, IGF-1 or any suitable IGF-1 receptor agonist may be administered in addition to or as an alternative to insulin and may have the same or similar effect as insulin.

Fluorine-18 Fluorodeoxyglucose Positron Emission Tomography (FDG-PET) Use in Metabolic Targeted Chemo-Immunotherapy

FDG-PET is a commonly used scanning technology for oncology based on positron emitting isotope radiation. Combined with x-ray based computed tomography (CT), PET/CT is capable of three dimensional anatomical imaging overlaid with biochemical based scanning. Based on the Warburg effect, FDG-PET is able to detect cancers which uptake glucose more readily than surrounding tissue. This ratio between normal tissue glucose uptake and cancer cell glucose uptake is calculated into a Standard Uptake Value (SUV) where a high value SUV correlates with a high glucose metabolism.

An FDG-PET scan presenting a lesion with a high SUV is indicative of malignancy of various cancers and is useful in staging of cancers. Traditionally, while an initial high SUV is predictive of malignancy and able to find metastasis, it not predictive of treatment outcome. A second FDG-PET scan post treatment with traditional chemo-radiation therapies is usually necessary to predict efficacy of treatment. (Workman 2006) (Weber 2005)

Because the Metabolic Targeted Chemo-Immunotherapy described herein specifically targets cancer cells and CSCs, which use high levels of glucose, initial FDG-PET scanning is predictive of our treatment outcome. Lesions with relative high SUV values, which can vary between different machines and sites performing the scan but generally greater than 2.5 SUV, will be susceptible to our treatment. These high glucose using lesions will respond well to Metabolic Targeted Chemo-Immunotherapy causing regression in those lesions.

In some embodiments, the methods Metabolic Targeted Chemo-Immunotherapy described herein preferentially targets a population of cancer cells over healthy cells. The population of cancer cells may be a population of cancer stem cells having a high standard uptake value (SUV).

In one embodiment, a fractionated dose metabolic targeted chemotherapy protocol is provided. Such a protocol may include the following steps:
1. administering a drug combination that includes Metformin at a dose of approximately 250 mg-2500 mg oral daily, taken throughout treatment.
2. fasting a patient overnight to reach baseline blood glucose levels, typically about 4.5-5.5 mmol/L for non-diabetic patients. Diabetic patients may have fasting blood glucose levels well above 14 mmol/L.
3. administering or injecting, on day 2, an i.v. with 500 cc saline. Intravenous insulin (Humalog) is injected into the patients based on their body weight to reduce blood glucose levels to roughly half baseline at about 2.2-2.8 mmol/L for a non-diabetic patient. Typically the dosage of insulin is 0.1 to 0.5 units/kg and the average is 0.2 units/kg. Blood glucose levels are monitored for duration of treatment.
4. administering or injecting each chemotherapy drug is injected separately (slow bolus), after the desired blood sugar level is reached. This includes drugs, such as cisplatin, that are generally administered by dripping saline. The dosage of each drug is typically approximately 5-25% of the standard chemotherapy dosage.
5. administering or injecting, after the chemotherapy treatment, intravenous injection of a glucose solution, typically 50 ml of 20% to recover from the low glucose levels; and rehydrating the patient with saline and consumption of liquids.
6. patients are typically treated once, twice or three times per week with chemotherapy. Patients will normally receive 12-24 total treatments, though this number may vary due to clinical outcome or various other factors.

Synergy Between Metabolic Targeting Chemotherapy and Immunologics.

The idea that malignant cancer cells retain stem cell characteristics because they come from fusion between stem cells and non-malignant cells suggests it is also possible to turn malignant cancer cells into antigen-presenting cells (APCs) which makes them detectable by the immune system (FIG. 13).

Treatment of mesenchymal stem cells (MSCs) with interferon-Gamma causes them to become APCs (Chan et al). Other APCs (ie. Dendritic cells) can be derived by treating monocytes progenitor cells with GM-CSF, IL-4 and TNF-alpha (Ohgimoto et al.). Therefore, according to some embodiments, the methods described herein may include treating malignant cancer cells with interferon-Gamma, causing them to become APCs' and treating malignant cancer cells with GM-CSF, IL-4 and TNF-alpha, causing the cells to turn into dendritic cell-like APCs.

Immunotherapy and Immunologic Targeting Treatment

In one embodiment, a method for treating cancer may include an immunologic targeting treatment regimen. The immunologic targeting treatment regimen may include the administration of one or more immunologic agent or immunotherapy.

In some embodiments, metabolic targeting chemotherapeutic treatments as described above may, in effect, be considered an immunotherapy due to its immune cell sparing effect. By reducing the damage to the immune system and its innate ability to target cancer cells, an immunotherapeutic effect is achieved when compared to typical chemotherapy or combination chemotherapy.

Immunologic agents can have direct cytotoxic and/or immunological effects against tumor cells. They are often administered in conjunction with chemotherapy in treatment of cancers. However, standard doses of cancer chemotherapy have significant immune toxicity. This makes the therapeutic efficacy of chemotherapy and immunologics difficult to combine.

As described above, a metabolic targeting chemotherapy regimen may include reducing a patient's blood glucose level and administering a therapeutically effective dose one or more chemotherapeutic agents, wherein the therapeutically effective dose is lower than a standard dose of chemotherapy. By using chemotherapy drugs at lower than standard doses, the cells of the immune system will be, in large part, spared. Thus, an immune response against opportunistic infections and the cancer itself may be more effectively induced by administering an immunologic targeting treatment regimen in combination with the metabolic targeting chemotherapy regimen. The combination of low-dose chemotherapy with immunotherapy will result in a synergistic therapeutic effect.

Therefore, in some embodiments, a method for treating cancer may include administering a metabolic targeting chemotherapy treatment regimen in combination with an immunologic targeting treatment regimen. The immunologic targeting treatment regimen may include administering a therapeutically effective dose of one or more immunologic agents to stimulate an immune response in a subject having cancer. The immunologic agents may include any of the agents described herein, including pegylated forms thereof, liposomal forms thereof and any other suitable modified forms.

There are two main types of immunologic agents, active and passive. Active immunologic agents, such as vaccines, stimulate an immune response to one or more specific antigenic types. In contrast, passive immunologic agents do not have antigenic specificity but can act as general stimulants that enhance the function of certain types of immune cells. Immunologic agents that may be used in an immunologic targeting treatment regimen may include immunostimulant substances that modulate the immune system by stimulating the function of one or more of the system's components. The methods described herein may be combined with behaviors or treatments that may stimulate the immune system including, but not limited to, exercise, meditation, yoga, deep breathing, tai chi/Qigong, and acupuncture.

In some embodiments, immunologic agents that may be used in accordance with the methods described herein include, but are not limited to, vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies.

In some embodiments, the one or more immunologic agents may include, but is not limited to, vitamin C, vitamin A, vitamin E, vitamin B-6), carotenoids and beta carotene, selenium, zinc, flavanoids and bioflavanoids, iron chelators, astragalus, beta-glucans, echinacea, elderberry, garlic, ginger, ginseng, ganoderma lucidum (Reishi or Ling Zhi), medicinal mushrooms (Reishi or Agaricus blazei), bee propolis, snake venom, scorpion, colostrum (e.g., bovine colostrum), indirubin, cordycepssinensis, scutellaria baicalensis georgi, rhemannia glutinosa (Chinese Foxglove, Shen di Huang), quercetin, coenzyme Q10, lysine carnitine, glutathione-containing compounds, omega-3 fatty acids, prolactin, growth hormone, alpha-lipoic acid, lentinan, polysaccharide-K (MC-S), synthetic cytosine phosphate-guanosine (CpG), oligodeoxynucleotides, interleukins (e.g., IL-2 or IL-12), tumor necrosis factor alpha or beta (TNF-α or -β), granulocyte colony-stimulating factor (G-CSF), B7-1, ICAM-1, LFA-3, proline-rich polypeptides (PRPs, which can be made or derived from mammalian cololstrum such as bovine colostrum), imiquimod, beta-glucans, BCG vaccine, tumor antigens, killed tumor cell therapy, gene therapy vectors expressing cytokines, T cell costimulatory molecules or other suitable immunostimulatory molecules, dendritic cell based immunotherapeutics, T cell based adoptive immunotherapeutics.

In other embodiments, the one or more immunologic agent used in the methods described herein may be a therapeutic antibody or a functional fragment thereof that targets cancer cells. Passive immunotherapy in the form of therapeutic antibodies has been the subject of considerable research and development as anti-cancer agents. Therapeutic antibodies are typically administered in maximum tolerated doses to block target receptors that are overexpressed on cancer cells, blocking the receptor's function systemically. However, given at a dose that is substantially lower than the maximum tolerated dose (e.g., ½ to 1/1000th of the standard dose) allows the therapeutic antibody to act as an immunostimulant. After binding a target cancer cell, therapeutic antibodies or functional fragments thereof may stimulate cytotoxic immune-mediated responses, such as antibody-dependent cell-mediated cytotoxicity and complement-dependent cytotoxicity, mediated by Fc region activation of complement or Fc receptor (FcR) engagement. After cancer cells have been lysed, macrophages and other phagocytic, antigen presenting immune cells may engulf the components of the lysed cell and present cancer cell antigens to stimulate an acquired immune response against the cancer cells.

Examples of therapeutic antibodies that may be used as an immunologic agent according to the embodiments of the disclosure include, but are not limited to, alemtuzumab, bevacizumab, cetuximab, edrecolomab, gemtuzumab, ibritumomab tiuxetan, panitumumab, rituximab, tositumomab, and trastuzumab.

The immunologic agent and/or chemotherapeutic agent used in the treatment regimens described herein may be administered to a patient as part of a pharmaceutical composition or formulation. In some embodiments, the pharmaceutical composition or formulation may include one or more immunologic agent, one or more chemotherapeutic agent, a physiologically acceptable carrier, or a combination thereof.

In some embodiments, the treatment regimens described herein may be used before, after or in combination with other cancer therapies, including, but not limited to, surgery, cryosurgery, light therapy and hyperthermia therapy.

Having described the invention with reference to the embodiments and illustrative examples, those in the art may appreciate modifications to the invention as described and illustrated that do not depart from the spirit and scope of the invention as disclosed in the specification. The examples are set forth to aid in understanding the invention but are not intended to, and should not be construed to limit its scope in any way. The examples do not include detailed descriptions of conventional methods. Such methods are well known to those of ordinary skill in the art and are described in numerous publications. In addition, all of the references cited in the disclosure are hereby incorporated in their entirety by reference as if fully set forth herein.

### Example 1: Metabolic Targeting Chemotherapy Has a Beneficial Clinical Effect

Patients. Six patients having various metastatic late stage cancers (i.e., stage IIIB/IV) that were previously unresponsive to standard treatment with one or more chemotherapeutic agents, radiation, surgery or other modalities of treatment were selected and underwent treatment as described below.

Treatment Regimen. Prior to treatment, patients were imaged by computed tomography (CT), magnetic resonance imaging (MM), positron emission tomography (PET), or a combination thereof, and a baseline tumor mass was calculated. On day 0, patients were fasted overnight to reach baseline glucose levels (typically about 4.5-5.5 mmol/L). The following day (Day 1), after an initial intravenous flush with 500 cc saline, all patients received insulin (Humalog) at a dose of 0.1 to 0.5 units/kg body weight intravenously (i.v.) to lower blood glucose levels to roughly half of the baseline level (about 2.2-2.8 mmol/L). Blood glucose levels were monitored for the duration of the subsequent treatment regimen.

After the desired blood glucose levels were reached, one or more chemotherapeutic agents were injected (i.v.) separately by a slow bolus or by dripping saline at an initial dosage that is 5-50% of the standard dosage for each particular agent. The dosage may be increased according to the patient's response to the initial dose or disease progression. After the chemotherapeutic treatment, patients were recovered from low blood glucose levels with an i.v. injection of a glucose solution (typically 50 ml of 20% glucose solution) and were rehydrated with saline and consumption of liquids.

Patients were treated once or twice weekly with the treatment regimen described above for up to 18 total treatments. Table 2 (shown below) shows information and the specific chemotherapeutic agents used in the treatment regimen for each patient.

TABLE 2
Treatment Regimens

| Patient | | Type of | | # of |
|---|---|---|---|---|
| No | Age/Sex/Weight | Cancer | Chemotherapeutics used (Dosage) | Treatments |
| 1 | 56/M/62 kg | Esophageal | Gemcitabine (400 mg) + THP- | 12 |
| | | | COHP* (30 mg) + 5-fluorouracil | |
| | | | (200 mg) | |
| | | | Gemcitabine (200 mg) + THP- | 6 |
| | | | COHP* (25 mg) + 5-fluorouracil | |
| | | | (200 mg) | |
| | | | | 18 (TOTAL) |
| 2 | 55/M/50 kg | Colon | COHP* (50 mg) + 5-fluorouracil | 12 |
| | | | (200 mg) | |
| | | | Mitomysin C (2 mg) + vincristine | 6 |
| | | | (0.5 mg) + 5-fluorouracil (200 mg) | |
| | | | | 18 (TOTAL) |
| 3 | 51/F/60 kg | Breast | Cyclophosphamide (200 mg) + | 6 |
| | | | cisplatin (10 mg) + doxorubicin | |
| | | | (6 mg) | |
| | | | | 6 (TOTAL) |
| 4 | 30/F/47 kg | Lung | Cyclophosphamide (100 mg) + | 6 |
| | | | vinorelbine (6 mg) | |
| | | | | 6 (TOTAL) |
| 5 | 72/M/47 kg | Esophagus | Etoposide (30 mg) + oxaliplatin | 12 |
| | | | (30 mg) | |
| | | | Methotrexate (10 mg) + oxaliplatin | 6 |
| | | | (30 mg) + gemcitabine (200 mg) | |
| | | | | 18 (TOTAL) |
| 6 | 47/F/50 kg | Stomach | Etoposide (20 mg) + pirarubicin | 18 |
| | | | (8 mg) + 5-fluorouracil (200 mg) | |
| | | | | 18 (TOTAL) |

Evaluation of Tumor Response.

After treatment, patients were imaged by computed tomography (CT), magnetic resonance imaging (MM), positron emission tomography (PET), or a combination thereof, to calculate a post-treatment tumor mass. A response category was assigned to each patient based on a comparison of the baseline tumor mass to the post-treatment tumor mass.

The Standard World Health Organization (WHO) criteria were used to determine the response to the treatment regimen described above. Briefly, a complete response (CR) category is assigned when no clinically detectable cancer is found after treatment. A partial response (PR) category is assigned when a decrease in measurable tumor mass is observed (≥50% decrease), no new areas of tumor develop, and no area of tumor shows progression. A minimal response (MR) category is assigned when a decrease in measurable tumor mass is observed (<50% decrease), no new areas of tumor develop, and no area of tumor shows progression. A progressive disease (PD) category is assigned when the mass of one or more tumor sites increases (>25% increase) or new lesions appear. A stable disease (SD) category is assigned when a measurable mass does not meet the criteria for CR, PR, MR or PD. A patient is considered to have received a clinical benefit as a result of a particular treatment regimen based an objective response of CR, PR, MR, or SD.

Table 3 shows tumor response to the treatment regimens described above. Briefly, patients 5 and 6 showed a partial response, patients 2 and 3 showed a minimal response, patient 1 showed stable disease and patient 4 showed progressive disease. Based on these results, ⅚ showed a clinical benefit of metabolic targeting therapy. This indicates that a treatment regimen that includes a decrease in blood glucose by administering a dose of insulin in combination with one or more chemotherapeutic agents results in an improvement over treatment with one or more chemotherapeutic agents alone in patients with late stage cancer.

TABLE 3
Response to Treatment

| | | | Assigned | |
|---|---|---|---|---|
| Patient | | Type of | Response | Clinical |
| No | Age/Sex/Weight | Cancer | Category | Benefit? |
| 1 | 56/M/62 kg | Esophageal | SD | Yes |
| 2 | 55/M/50 kg | Colon | MR | Yes |
| 3 | 51/F/60 kg | Breast | MR | Yes |
| 4 | 30/F/47 kg | Lung | PD | No |
| 5 | 72/M/47 kg | Esophagus | PR | Yes |
| 6 | 47/F/50 kg | Stomach | PR | Yes |

### Example 2: Metabolic Targeting Chemo-Immunotherapy for the Treatment of Cancer

Patients.

Patients treated at Xi'an Xingcheng Borui Hospital ( [symbol] [symbol] ) and Yangling Demonstration Zone Hospital ( [symbol] ), having various metastatic late stage cancers (i.e., stage IIIB/IV; cancer type shown in Table 4 below) and were previously unresponsive to standard treatment with one or more chemotherapeutic agents, radiation, surgery or other modalities of treatment were selected to undergo treatment as described below.

Treatment Regimen.

Prior to treatment, patients were imaged by computed tomography (CT), magnetic resonance imaging (MM), positron emission tomography (PET), or a combination thereof, and a baseline tumor mass was calculated.

On day 0, patients were administered an immunologic agent such as a therapeutic antibody or functional fragment thereof. Therapeutic antibodies that will be used in this treatment regimen may include commercially available antibodies against EGFR or HER2 such as cetuximab (Erbitux®), panitumumab (Vectibix®) and trastuzumab (Herceptin®). The therapeutic antibody was administered to the patients at a dose that is lower than the standard dose typically given for cancer treatment. The dose is typically between about ½ to 1/1000th of the standard dose. Although a single dose of the therapeutic antibody may be sufficient for the duration of the therapeutic regimen, optional additional booster doses may be given if needed. The booster dose may be repeated every two weeks, if needed.

Following treatment with the immunologic agent, the patients were fasted overnight to reach baseline glucose levels (typically about 4.5-5.5 mmol/L). The following day (Day 1), after an initial intravenous flush with 500 cc saline, all patients received insulin (Humalog) at a dose of 0.1 to 0.5 units/kg body weight intravenously (i.v.) to lower blood glucose levels to roughly half of the baseline level (about 2.2-2.8 mmol/L). Blood glucose levels are monitored for the duration of the subsequent treatment regimen.

After the desired blood glucose levels are reached, one or more chemotherapeutic agents will be injected (i.v.) separately by a slow bolus or by dripping saline at a dosage that is 5-25% of the standard dosage for each particular agent. The one or more chemotherapeutic agents were selected based on the type of cancer and severity of the disease. For example, the agents may be any one or more chemotherapeutic agents in combination as described in Example 1 above (see patents 1-6 in Table 2), or may be one or more of the agents described in the disclosure above. After the chemotherapeutic treatment, patients will be recovered from low blood glucose levels with an injection (i.v.) of a glucose solution (typically 50 ml of 20% glucose solution) and are rehydrated with saline and consumption of liquids.

Patients were treated once or twice weekly with the treatment regimen described above for up to 24 total treatments. The total number of treatments may vary due to clinical outcome or various other factors as determined by one skilled in the art. Table 4 below summarizes the treatment regimens used on each of the patients.

TABLE 4
Treatment Regimens

| Patient | | Type of | Chemotherapeutics used | # of | Erbitux |
|---|---|---|---|---|---|
| No | Age/Sex/Weight | Cancer | (Dosage) | Treatments | (Dosage) |
| 1 | 71/M/68 kg | Lung | Vinorelbine (10 mg) + | 12 | 1 × 20 mg |
| | | | Cisplatin (10 mg) | | |
| 2 | 60/M/63 kg | Stomach | Vinorelbine (10 mg) + | 12 | 1 × 20 mg |
| | | | Cisplatin (10 mg) + | | |
| | | | 5-fluorouracil (250 mg) | | |
| 3 | 52/F/49 kg | Stomach | Vinorelbine (10 mg) + | 12 | 1 × 20 mg |
| | | | Cisplatin (10 mg) + | | |
| | | | 5-fluorouracil (250 mg) | | |
| 4 | 43/F/53 kg | Breast | Vinorelbine (10 mg) + | 12 | 1 × 20 mg |
| | | | Epirubicin (10 mg) | | |
| 5 | 47/M/70 kg | Esophagus | Vinorelbine (10 mg) + | 12 | 1 × 20 mg |
| | | | Cisplatin (10 mg) + | | |
| | | | 5-fluorouracil (250 mg) | | |
| 6 | 62/M/65 kg | Lung | Etoposide (100 mg) + | 12 | 1 × 20 mg |
| | | | Cisplatin (10 mg) | | |

Evaluation of Tumor Response. After treatment, patients were imaged by computed tomography (CT), magnetic resonance imaging (MM), positron emission tomography (PET), or a combination thereof, to calculate a post-treatment tumor mass. A response category will be assigned to each patient based on a comparison of the baseline tumor mass to the post-treatment tumor mass.

The Standard World Health Organization (WHO) criteria will be used to determine the response to the treatment regimen described above. Briefly, a complete response (CR) category is assigned when no clinically detectable cancer is found after treatment. A partial response (PR) category is assigned when a decrease in measurable tumor mass is observed (≥50% decrease), no new areas of tumor develop, and no area of tumor shows progression. A minimal response (MR) category is assigned when a decrease in measurable tumor mass is observed (<50% decrease), no new areas of tumor develop, and no area of tumor shows progression. A progressive disease (PD) category is assigned when the mass of one or more tumor sites increases (>25% increase) or new lesions appear. A stable disease (SD) category is assigned when a measurable mass does not meet the criteria for CR, PR, MR or PD. A patient is considered to have received a clinical benefit as a result of a particular treatment regimen based an objective response of CR, PR, MR, or SD.

Table 5, below shows the response of each patient to the treatment regimen described above.

TABLE 5
Response to Treatment

| | | | Assigned | |
|---|---|---|---|---|
| Patient | | Type of | Response | Clinical |
| No | Age/Sex/Weight | Cancer | Category | Benefit? |
| 1 | 71/M/68 kg | Lung | CR | Yes |
| 2 | 60/M/63 kg | Stomach | CR | Yes |
| 3 | 52/F/49 kg | Stomach | PR | Yes |
| 4 | 43/F/53 kg | Breast | PR | Yes |
| 5 | 47/M/70 kg | Esophagus | PR | Yes |
| 6 | 62/M/65 kg | Lung | PR | Yes |

Results will be compared to the results in Example 1. Briefly, all patients showed a response and clinical benefit as a result of receiving the treatment regimen: two of which showed a complete response. Thus, patients treated with one or more immunologic agent (e.g., a therapeutic antibody) in addition to a metabolic targeting therapy show an increased clinical benefit as compared to the metabolic targeting therapy alone. This increased clinical benefit may be a result of a synergistic effect of the metabolic targeting of the cancer cells in combination with the low dose chemotherapeutic agents allowing a more efficient immune response to the Fc domain of the therapeutic antibodies.

Notably, Patient 4 showed a significant response to the treatment regimens described above. Patient 4 is a 43 year old female patient who presented with recurrent breast cancer 7 years post surgery and chemotherapy treatment. Wide spread metastasis was seen throughout the bones of the patient on a PET/CT scan (FIG. 14). The Patient was administered 12 treatments of metabolic targeted chemo-immunotherapy over a period of 6 weeks. A PET/CT scan taken after treatment showed remarkable remission of bone metastasis (FIG. 14, top panel).

The same patient presented with a large mass in the lower left lung measuring 1.3×1.5×0.8 cm in the pre-treatment PET/CT scan (FIG. 15, bottom panel). SUV value was low, 1.5, indicating low glucose uptake in the lesion. A follow up PET/CT scan showed an increase of the left lung mass size, 1.3×1.5×1.8 cm, however a slight decrease in SUV, 1.3 (FIG. 15, top panel), indicating that although the size increased, the metabolic state of the lesion was likely more stable, which may contribute to a reduction in the aggressiveness of the cancer. The results of these studies indicate that the metabolic targeting chemo-immunotherapy regimens described herein target metastatic cancer cells, in particular, the treatment methods described herein target cancer stem cells, which are likely the predominant cancer cell type responsible for metastasis and invasiveness of a cancer.

According to an embodiment,

1. A method for treating cancer comprising administering a metabolic targeting chemo-immunotherapy regimen, the metabolic targeting chemo-immunotherapy regimen comprising:

administering a therapeutically effective dose of one or more immunologic agents to stimulate an immune response in a subject having cancer;

reducing the patient's blood glucose level; and

administering a therapeutically effective dose of one or more chemotherapeutic agents;

wherein the therapeutically effective dose of the one or more immunologic agents and the one or more chemotherapeutic agents is lower then a standard dose.

2. The method of item 1, wherein the one or more immunologic agents are selected from the group consisting of vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies

3. The method of item 1, wherein at least one of the one or more immunologic agents is a therapeutic antibody or functional fragment thereof selected from the group consisting of alemtuzumab, bevacizumab, cetuximab, edrecolomab, gemtuzumab, ibritumomab tiuxetan, panitumumab, rituximab, tositumomab, and trastuzumab.

4. The method of item 3, further comprising administering one or more booster doses of the one or more therapeutic antibodies.

5. The method of item 4, wherein the one or more booster doses are administered at an interval of two weeks.

6. The method of item 1, wherein the immunologic agent is selected from interferon-Gamma, GM-CSF, IL-4 or TNF-alpha and administration of said immunologic agent causes the cancer cells to become antigen presenting cells.

7. The method of item 1, wherein the immune response is a specific immune response.

8. The method of item 1, wherein the immune response is a non-specific immune response.

9. The method of item 1, wherein the blood glucose level is reduced by fasting, administering a dose of insulin, administering a dose of an insulin independent agent, or a combination thereof.

10. The method of item 9, wherein the insulin-independent agent is metformin, glimepiride, glyburide or glipizide.

11. The method of item 1, wherein the reduction in blood sugar is maximized.

12. The method of item 1, wherein the reduction in blood sugar is maintained for a prolonged period of time.

13. The method of item 1, further comprising administering a therapeutically effective dose of an insulin-independent agent in combination with the one or more chemotherapeutic agents.

14. The method of item 13, wherein the insulin-independent agent is administered during the administration of the chemotherapeutic agent, after the administration of the chemotherapeutic agent, or both.

15. The method of item 14, wherein the insulin-independent agent is metformin.

16. The method of item 1, wherein the one or more chemotherapeutic agents are selected from the group consisting of alkylating agents, antimetabolites, anti-tumor antibiotics, topoisomerase inhibitors, mitotic inhibitors hormone therapy, glycolysis inhibitors, targeted therapeutics and immunotherapeutics.

17. The method of item 1, wherein the one or more chemotherapeutic agents comprise a plurality of chemotherapeutic agents, each targeting a different point in the cell cycle.

18. The method of item 1, wherein the one or more chemotherapeutic agents are administered at a high frequency.

19. The method of item 1, wherein the metabolic targeting chemo-immunotherapy is used for treating a cancer selected from the group consisting of bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer.

20. The method of item 1, wherein the metabolic targeting chemo-immunotherapy targets malignant and metastatic cancer cells and cancer stem cells.

21. The method of item 1, wherein the metabolic targeting chemo-immunotherapy preferentially targets a population of cancer cells over healthy cells

22. The method of item 18, wherein the population of cancer cells is a population of cancer stem cells having a high standard uptake value (SUV).

23. A method for treating cancer comprising administering a metabolic targeting chemo-immunotherapy regimen, the metabolic targeting chemo-immunotherapy regimen comprising:

administering an initial therapeutically effective dose of a therapeutic antibody or functional fragment thereof to target a population of cancer cells and to stimulate an immune response in a subject having cancer;

reducing the patient's blood glucose level by fasting and/or administering a dose of insulin; and

administering a therapeutically effective dose of one or more chemotherapeutic agents;

wherein the therapeutically effective dose of the therapeutic antibody and the one or more chemotherapeutic agents is lower then a standard dose.

24. The method of item 23, wherein the one or more therapeutic antibodies are selected from the group consisting of alemtuzumab, bevacizumab, cetuximab, edrecolomab, gemtuzumab, ibritumomab tiuxetan, panitumumab, rituximab, tositumomab, and trastuzumab.

25. The method of item 23, further comprising administering one or more booster doses of the one or more therapeutic antibodies.

26. The method of item 25, wherein the one or more booster doses are administered at an interval of two weeks.

27. The method of item 23, wherein the immune response is a specific immune response.

28. The method of item 23, wherein the immune response is a non-specific immune response.

29. The method of item 23, wherein the reduction in blood sugar is maximized.

30. The method of item 23, wherein the reduction in blood sugar is maintained for a prolonged period of time.

31. The method of item 23, wherein the one or more chemotherapeutic agents are selected from the group consisting of alkylating agents, antimetabolites, anti-tumor antibiotics, topoisomerase inhibitors, mitotic inhibitors hormone therapy, glycolysis inhibitors, targeted therapeutics and immunotherapeutics.

32. The method of item 23, wherein the one or more chemotherapeutic agents comprise a plurality of chemotherapeutic agents, each targeting a different point in the cell cycle.

33. The method of item 23, wherein the one or more chemotherapeutic agents are administered at a high frequency.

34. The method of item 23, wherein the metabolic targeting chemo-immunotherapy is used for treating a cancer selected from the group consisting of bone cancer, bladder cancer, brain cancer, breast cancer, cancer of the urinary tract, carcinoma, cervical cancer, colon cancer, esophageal cancer, gastric cancer, head and neck cancer, hepatocellular cancer, liver cancer, lung cancer, lymphoma and leukemia, melanoma, ovarian cancer, pancreatic cancer, pituitary cancer, prostate cancer, rectal cancer, renal cancer, sarcoma, testicular cancer, thyroid cancer, and uterine cancer.

35. The method of item 23, wherein the metabolic targeting chemo-immunotherapy targets malignant and metastatic cancer cells and cancer stem cells.

36. The method of item 23, wherein the metabolic targeting chemo-immunotherapy preferentially targets a population of cancer cells over healthy cells

37. The method of item 36, wherein the population of cancer cells is a population of cancer stem cells having a high standard uptake value (SUV).

38. The method of item 23, further comprising administering an immunologic agent concurrently with the metabolic targeting chemo-immunotherapy

39. The method of item 38, wherein the immunologic agent is selected from the group consisting of vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies.

40. The method of item 23, wherein the immunologic agent is selected from interferon-Gamma, GM-CSF, IL-4 or TNF-alpha and administration of said immunologic agent causes the cancer cells to become antigen presenting cells.

41. A method for treating cancer comprising administering a metabolic targeting chemo-immunotherapy regimen, the metabolic targeting chemo-immunotherapy regimen comprising the steps of:

a) administering an initial therapeutically effective dose of one or more therapeutic antibodies to a subject having cancer to stimulate an immune response;

b) fasting the subject overnight;

c) administering an effective dose of insulin to the subject to reduce the subject's blood glucose level; and

d) administering a therapeutically effective dose of one or more chemotherapeutic agents;

wherein the therapeutically effective dose of the one or more immunologic agents and the one or more chemotherapeutic agents is lower then a standard dose.

42. The method of item 41, wherein the one or more therapeutic antibodies are selected from the group consisting of alemtuzumab, bevacizumab, cetuximab, edrecolomab, gemtuzumab, ibritumomab tiuxetan, panitumumab, rituximab, tositumomab, and trastuzumab.

43. The method of item 41, further comprising administering one or more booster doses of the one or more therapeutic antibodies.

44. The method of item 43, wherein the one or more booster doses are administered at an interval of two weeks.

45. The method of item 41, wherein the reduction in blood sugar is maximized.

46. The method of item 41, wherein the reduction in blood sugar is maintained for a prolonged period of time.

47. The method of item 41, wherein the one or more chemotherapeutic agents are selected from the group consisting of alkylating agents, antimetabolites, anti-tumor antibiotics, topoisomerase inhibitors, mitotic inhibitors hormone therapy, glycolysis inhibitors, targeted therapeutics and immunotherapeutics.

48. The method of item 47, wherein the one or more chemotherapeutic agents comprise a plurality of chemotherapeutic agents, each targeting a different point in the cell cycle.

49. The method of item 47, wherein the one or more chemotherapeutic agents are administered at a high frequency.

50. The method of item 41, wherein the metabolic targeting chemo-immunotherapy targets malignant and metastatic cancer cells and cancer stem cells.

51. The method of item 41, wherein the metabolic targeting chemo-immunotherapy preferentially targets a population of cancer cells over healthy cells

52. The method of item 51, wherein the population of cancer cells is a population of cancer stem cells having a high standard uptake value (SUV).

53. A method for treating cancer comprising administering a metabolic targeting chemotherapy regimen, the metabolic targeting chemotherapy regimen comprising:

reducing a cancer patient's blood glucose level in an insulin independent manner to target MDR pumps expressed in a population of cancer stem cells; and

administering a therapeutically effective dose of one or more chemotherapeutic agents, wherein the therapeutically effective dose of the one or more chemotherapeutic agents is lower than a standard dose.

54. The method of item 53, wherein the insulin independent manner of reducing the blood glucose level is selected from fasting, exercise, low carbohydrate diet, administration of an alpha-glucosidase inhibitor, administration of a biguanide drug, or a combination thereof.

55. The method of item 53, wherein the reduction in blood sugar is maximized.

56. The method of item 53, wherein the reduction in blood sugar is maintained for a prolonged period of time.

57. The method of item 53, wherein the one or more chemotherapeutic agents are selected from the group consisting of alkylating agents, antimetabolites, anti-tumor antibiotics, topoisomerase inhibitors, mitotic inhibitors hormone therapy, glycolysis inhibitors, targeted therapeutics and immunotherapeutics.

58. The method of item 53, wherein the one or more chemotherapeutic agents comprise a plurality of chemotherapeutic agents, each targeting a different point in the cell cycle.

59. The method of item 53, wherein the one or more chemotherapeutic agents are administered at a high frequency.

60. The method of item 53, further comprising administering a therapeutically effective dose of an MDR pump inhibitor.

61. The method of item 60, wherein the MDR pump inhibitor is selected from the group consisting of verapamil, quinidine, quinine, cyclosporine A, PSC 833, VX-710, LY335979, R101933, OC144-093 and XR9576.

62. The method of item 53, further comprising administering a therapeutically effective dose of one or more immunologic agents to stimulate an immune response, wherein the therapeutically effective dose of the one or more chemotherapeutic agents is lower than a standard dose.

63. The method of item 62, wherein the one or more immunologic agents are selected from the group consisting of vitamins, minerals, nutrients, herbs, plant-derived substances, fungi, animal or insect-derived substances, adjuvants, antioxidants, amino acids, cytokines, chemokines, hormones, T cell costimulatory molecules, general immune-stimulating peptides, gene therapy, immune cell-derived therapy, and therapeutic antibodies.

64. The method of item 53, wherein the immunologic agent is selected from interferon-Gamma, GM-CSF, IL-4 or TNF-alpha and administration of said immunologic agent causes the cancer cells to become antigen presenting cells.

65. The method of item 53, wherein the metabolic targeting chemo-immunotherapy targets malignant and metastatic cancer cells and cancer stem cells.

66. The method of item 53, wherein the metabolic targeting chemo-immunotherapy preferentially targets a population of cancer cells over healthy cells

67. The method of item 66, wherein the population of cancer cells is a population of cancer stem cells having a high standard uptake value (SUV).

68. The method of item 53, further comprising administering a subsequent dose of insulin.

References in this description to “an embodiment”, “one embodiment”, or the like, mean that the particular feature, function, structure or characteristic being described is included in at least one embodiment of the present invention. Occurrences of such phrases in this specification do not necessarily all refer to the same embodiment. On the other hand, such references are not necessarily mutually exclusive either.

Note that any and all of the embodiments described above can be combined with each other, except to the extent that it may be stated otherwise above or to the extent that any such embodiments might be mutually exclusive in function and/or structure.

References in this description to “an embodiment”, “one embodiment”, or the like, mean that the particular feature, function, structure or characteristic being described is included in at least one embodiment of the present invention. Occurrences of such phrases in this specification do not necessarily all refer to the same embodiment. On the other hand, such references are not necessarily mutually exclusive either.

Note that any and all of the embodiments described above can be combined with each other, except to the extent that it may be stated otherwise above or to the extent that any such embodiments might be mutually exclusive in function and/or structure.

### REFERENCES

All of the references listed below and all of those cited in the specification above are hereby incorporated in their entirety by reference as if fully set forth herein.
Hirayama A, Kami K, Sugimoto M, Sugawara M, Toki N, Onozuka H, Kinoshita T, Saito N, Ochiai A, Tomita M, Esumi H. Quantitative metabolome profiling of colon and stomach cancer microenvironment by capillary electrophoresis time-of-flight mass spectrometry. Cancer research. 2009 Jun. 1; 69(11):4918-25.
Vaughn A E, Deshmukh M. Glucose metabolism inhibits apoptosis in neurons and cancer cells by redox inactivation of cytochrome c. Nature cell biology. 2008 Dec. 1; 10(12):1477-83.
Patra K C, Hay N. The pentose phosphate pathway and cancer. Trends in biochemical sciences. 2014 Aug. 31; 39(8):347-54.
Cao X, Fang L, Gibbs S, Huang Y, Dai Z, Wen P, Zheng X, Sadee W, Sun D. Glucose uptake inhibitor sensitizes cancer cells to daunorubicin and overcomes drug resistance in hypoxia. Cancer chemotherapy and pharmacology. 2007 Mar. 1; 59(4):495-505.
Maschek G, Savaraj N, Priebe W, Braunschweiger P, Hamilton K, Tidmarsh G F, De Young L R, Lampidis T J. 2-deoxy-D-glucose increases the efficacy of adriamycin and paclitaxel in human osteosarcoma and non-small cell lung cancers in vivo. Cancer research. 2004 Jan. 1; 64(1):31-4.
Ariës IM, Hansen B R, Koch T, van den Dungen R, Evans W E, Pieters R, den Boer M L. The synergism of MCL1 and glycolysis on pediatric acute lymphoblastic leukemia cell survival and prednisolone resistance. haematologica. 2013 Oct. 18:haematol-2013.
Tesori V, Piscaglia A C, Samengo D, Barba M, Bernardini C, Scatena R, Pontoglio A, Castellini L, Spelbrink J N, Maulucci G, Puglisi M A. The multikinase inhibitor Sorafenib enhances glycolysis and synergizes with glycolysis blockade for cancer cell killing. Scientific reports. 2015 Mar. 17; 5:9149.
Shin Y K, Yoo B C, Hong Y S, Chang H J, Jung K H, Jeong S Y, Park J G. Upregulation of glycolytic enzymes in proteins secreted from human colon cancer cells with 5-fluorouracil resistance. Electrophoresis. 2009 Jun. 1; 30(12):2182-92.
Helen H W C, Macus Tien K. Role of glutathione in the regulation of cisplatin resistance in cancer chemotherapy. Metal-based drugs. 2010 Sep. 14; 2010.
Doyle L A, Yang W, Abruzzo L V, Krogmann T, Gao Y, Rishi A K, Ross D D. A multidrug resistance transporter from human MCF-7 breast cancer cells. Proceedings of the National Academy of Sciences. 1998 Dec. 22; 95(26):15665-70.
Raez L E, Papadopoulos K, Ricart A D, Chiorean E G, DiPaola R S, Stein M N, Lima C M, Schlesselman J J, Tolba K, Langmuir V K, Kroll S. A phase I dose-escalation trial of 2-deoxy-D-glucose alone or combined with docetaxel in patients with advanced solid tumors. Cancer chemotherapy and pharmacology. 2013 Feb. 1; 71(2):523-30.
Zou K, XIE H. Pretreatment with insulin enhances anticancer functions of 5-fluorou-racil in human esophageal and colonic cancer cells. Acta pharmacologica sinica. 2007 May 1; 28(5):721-30.
Laitinen T, Lyyra-Laitinen T, Huopio H, Vauhkonen I, Halonen T, Hartikainen J, Niskanen L, Laakso M. Electrocardiographic alterations during hyperinsulinemic hypoglycemia in healthy subjects. Annals of Noninvasive Electrocardiology. 2008 Apr. 1; 13(2):97-105.
Ayala J E, Bracy D P, Malabanan C, James F D, Ansari T, Fueger P T, McGuinness O P, Wasserman D H. Hyperinsulinemic-euglycemic clamps in conscious, unrestrained mice. Journal of visualized experiments: JoVE. 2011 (57).
Abita, J. F., et al., 1984, Leukemia Res. 8:213.
Alabaster O., Vonderharr B. K., Shafie S. M. Metabolic modification by insulin enhances methotrexate cytoxicity in MCF-7 human breast cancer cells. Eur J Cancer Clin Oncol 1981; 17:1223-1228.
Al-Hajj M, Wicha M S, Benito-Hernandez A, Morrison S J, Clarke M F. Prospective identification of tumorigenic breast cancer cells. Proceedings of the National Academy of Sciences USA 100:3983-3988, 2003.
Ayre, S G, Skaletski B., Mosnaim A. D. Blood-brain barrier passage of azidothymidine in rats: effect of insulin. Res Commun Chem Pathol Pharmacol 1989; 63:45-52.
Ayre, S G, Perez Garcia y Bellon D., Perez Garcia Jr., D. Insulin potentiation therapy: a new concept in the management of chronic degenerative disease. Med Hypotheses 1986; 20(2):199-210.
Ayre, S G, Perez Garcia y Bellon D., Perez Garcia Jr., D. Neoadjuvant low-dose chemotherapy with insulin in breast carcinomas. Eur J Cancer 1990; 26: 1262-1263.
Ayre, S G, et al., 2000, Medical Hypotheses 55:330.
Ayre, S G, New approaches to the delivery of drugs to the brain. Med Hypotheses 1989; 29:283-291.
Bonilla L, Ben-Aharon I, Vidal L, Gafter-Gvili A, Leibovici L, Stemmer S M., “Dose-dense chemotherapy in nonmetastatic breast cancer: a systematic review and meta-analysis of randomized controlled trials.” J Natl Cancer Inst. 2010 Dec. 15; 102(24):1845-54.
Chan, Jennifer L., Katherine C. Tang, Anoop P. Patel, Larissa M. Bonilla, Nicola Pierobon, Nicholas M. Ponzio and Pranela Rameshwar, Antigen-presenting property of mesenchymal stem cells occurs during a narrow window at low levels of interferon-Gamma □□. Blood 107: 4817-4824, 2006.
Colman P. G., Harrison L. C. Structure of insulin/insulin-like growth factor-1 receptors on the insulinoma cell, RIM-m5F. Biochem Biophys Res Commun 1984; 124:657-662.
Cullen J. K., Yee D., Sly W. S., et al. Insulin-like growth factor receptor expression and function in human breast cancer. Cancer Res 1990; 50:48-53.
Dean M, Fojo T, Bates S. Tumour stem cells and drug resistance. Nature Reviews Cancer 5:275-284, 2005.
Dean M. Cancer stem cells: Implications for Cancer Causation and Therapy Resistance, Discovery Medicine, 5(27):278-282, 2005
Dowling R J, Goodwin P J, Stambolic V., “Understanding the benefit of metformin use in cancer treatment.” BMC Med. 2011 Apr. 6; 9:33.
Elstrom R L, Bauer D E, Buzzai M, Karnauskas R, Harris M R, Plas D R, Zhuang H, Cinalli R M, Alavi A, Rudin C M, Thompson C B., “Akt stimulates aerobic glycolysis in cancer cells.” Cancer Res. 2004 Jun. 1; 64(11):3892-9.
Emerman J. T, Siemiatkowski J. Effects of endocrine regulation of growth of a mouse mammary tumor on its sensitivity to chemotherapy. Cancer Res 1984; 44: 1327-1332.
Eppenberger U. New aspects in the molecular growth regulation of mammary tumors. In: Eppenberger U., Goldhirsch A. eds. Recent Results in Cancer Research, Vol. 113: Endocrine Therapy and Growth Regulation of Breast Cancer. Berlin-Heidelberg, 1989, 1-3.
Gasparro F P, et al. Receptor-mediated photo-cytotoxicity: synthesis of a photoactivatable psoralen derivative conjugated to insulin. Biochem Biophys Res Comm 1986; 141:502-509.
Gottesman M M, Fojo T, Bates S E. Multi-drug resistance in cancer: role of ATP-dependent transporters. Nature Reviews Cancer 2:48-58, 2002.
Goustin A. S., Leof E. B., Shipley G. D., Moses H. L. Growth factors and cancer. Cancer Res 1986; 46:1015-1029.
Gross G. E., Boldt D. H., Osborne C. K. Perturbation by insulin of human breast cancer cell kinetics. Cancer Res 1984; 44:3570-3575.
Xianghui He, Tom C. Tsang, Brain L. Pipes, Richard J. Ablin and David T. Harris, “A Stem Cell Fusion Model of Carcinogenesis” Journal of Experimental Therapeutics and Oncology, Vol. 5, pp. 101-109, 2005.
Heddleston J M, Li Z, Lathia J D, Bao S, Hjelmeland A B, Rich J N., “Hypoxia inducible factors in cancer stem cells.” Br J Cancer. 2010 Mar. 2; 102(5):789-95. Epub 2010 Jan. 26
Hirsch H A, Iliopoulos D, Tsichlis P N, Struhl K., “Metformin selectively targets cancer stem cells, and acts together with chemotherapy to block tumor growth and prolong remission.” Cancer Res. 2009 Oct. 1; 69(19):7507-11. Epub 2009 Sep. 14. Erratum in: Cancer Res. 2009 Nov. 15; 69(22):8832.
Hilf R. The actions of insulin as a hormonal factor in breast cancer. In: Pike M. C, Siiteri P. K, Welsch C. W, eds. Hormones and Breast Cancer, Cold Spring Harbor Laboratory, 1981: 317-337.
Holdaway I. M., Freisen H. G. Hormone binding by human mammary carcinoma. Cancer Research 1977; 37:1946-1952.
Jacobs S, Cook S, Svoboda M, Van Wyk J. J. Interaction of the monoclonal antibodies alpha-IR-1 and alpha-IR3 with insulin and somatomedin-C receptors. Endocrinol 1986; 118:223-226.
Jaques G., Rotsch M., Wegmann C., et al. Production of immunoreactive insulin-like growth factor 1 and response to exogenous IGF-1 in small cell lung cancer cell lines. Exp Cell Res 1988; 176: 336-343.
Jeffcoat R. and Jame A. T. The regulation of desaturation and elongation of fatty acids in mammals. Numa S. (Ed), Fatty Acid Metabolism and its Regulation. Elsevier Science Publishers BN. p. 85-112, 1984
Karey K. P., Sirbasku D. A. Differential responsiveness of human breast cancer cell lines MCF-7 and T47D to growth factors and 17B-estradiol. Cancer Res 1988; 48:4083-4092.
Kiang D. T., Bauer G. E., Kennedy B. J. Immunoassayable insulin in carcinoma of the cervix associated with hypoglycemia. Cancer 1973; 31:801-804.
Koroljow, S. Two cases of malignant tumors with metastases apparently treated successfully with hypoglycemic coma. Psychiatric Quarterly 1962; 36(1):261-270.
Lee P. D. K., Rosenfeld R. G., Hintz R. L., Smith S. D. Characterization of insulin, insulin-like growth factors I and II, and growth hormone receptors on human leukemic lymphoblasts. J Clin Endocr Metab 1986; 62: 28-35.
Leonard G D, Fojo T, Bates S E. The role of ABC trasporters in clinical practice. The Oncologist 2003; 8:411-424.
Lippman M. E., Dickson R. B., Kasid A., et al. Autocrine and paracrine growth regulation of human breast cancer. J Steroid Biochem 1986; 24:147-154.
Jeffcoat R. The biosynthesis of unsaturated fatty acids and its control in mammalian liver. Essays Biochem 1979; 15:1-36.
Poznansky M. J., Singh R., Singh B., Fantus G. Insulin: Carrier potential for enzyme and drug therapy. Science 1984; 223:1304-1306.
Mountjoy K. G., Holdaway I. M., Finlay G. J. Insulin receptor regulation in cultured human tumor cells. Cancer Research 1983; 43:4537-4542.
Myal Y., Shiu R. P. C., Bhomick B., Bala B. Receptor binding and growth promoting activity of insulin-like growth factors in human breast cancer cells [T-47D] in culture. Cancer Res 1984; 44:5486-5490.
Nakanishi Y., Mulshine J. L., Kasprzyk P. G., et al. Insulin-like growth factor-1 can mediate autocrine proliferation of human small cell lung cancer cell lines in vitro. J Clin Invest 1988; 82: 354-359.
Neufeld, O. Insulin therapy in terminal cancer: a preliminary report. J Amer Geriatric Soc 1962; 10(3):274-6.
Ohgimoto K, Ohgimoto S, Ihara T, Mizuta H, Ishido S, Ayata M, Ogura H, Hotta H. “Difference in production of infectious wild-type measles and vaccine viruses in monocyte-derived dendritic cells”. Virus Res 123 (1): 1-8, 2007.
Oleesky S., Bailey I., Samos S., Bilkus D. A fibrosarcoma with hypoglycemia and a high serum insulin level. Lancet 1962; 2:378-380.
Oster J. B., Creasey W. A. Enhancement of cellular uptake of ellipticine by insulin preincubation. Eur J Cancer Clin Oncol 1981; 17:1097-1103.
Papa V., Milazzo G., Goldfine I.D., Waldman F. M., Vigneri R. Sporadic amplification of the insulin receptor gene in human breast cancer. J Endocrinol Invest 1997; 20(9):531-6.
Papa V., Pezzino V., Costantino A., et al. Elevated insulin receptor content in human breast cancer. J Clin Invest 1990; 86:1503-1510.
Pardridge W. M., Eisenberg J., Yang J. Human blood-brain barrier insulin receptor. J Neurochem 1985; 44:1771-1778.
Pavelik L., Pavelik K., Vuk-Pavlovic S. Human mammary and bronchial carcinomas: in vivo and in vitro secretion of substances immunologically cross-reactive with insulin. Cancer 1984; 53(11):2467-2471.
Pavelik K., Bolanca M., Vecek N., et al. Carcinomas of the cervix and corpus uteri in humans: stage-dependent blood levels of substance(s) immunologically cross-reactive with insulin. J Natl Cancer Inst 1992; 68:891-894.
Pavelic K., Popovic M. Insulin and glucagon secretion by renal adenocarcinoma. Cancer 1981; 48:98-100.
Pavelic K., Odavic M., Pekic B., et al. Correlation of substance(s) immunologically cross-reactive with insulin, glucose and growth hormone in Hodgkin's lymphoma patients. Cancer Lett 1982; 17:81-86.
Pelicano H., Martin D S, Xu R-H, Huang P. Glycolysis inhibition for anticancer treatment. Oncogene 2006; 25:4633-4646.
Quinn K. A., Treston A. M., Unsworth E. J. et al. Insulin-like growth factor expression in human cancer cell lines. J Biol Chem 1996; 271:11477-83.
Raffaghello L, Lee C, Safdie F M, Wei M, Madia F, Bianchi G, Longo V D., “Starvation-dependent differential stress resistance protects normal but not cancer cells against high-dose chemotherapy.” Proc Natl Acad Sci USA. 2008 Jun. 17; 105(24):8215-20. Breast Cancer Res Treat 1998; 47(3):219-33.
Schilsky R. L., Bailey B. D., Chabner B. A. Characteristics of membrane transport of methotrexate by cultured human breast cancer cells. Biochem Pharmacol 1981; 30:1537-1542.
Shames J. M., Dhurandhar N. R., Blackard W. G. Insulin-secreting bronchial carcinoid tumor with widespread metastases. Am J Med 1968; 44:632-637.
Shinitzky M., Henkart P. Fluidity of cell membranes—current concepts and trends. Int Rev Cytol 1971: 60:121-147.
Simsek T, Kocabas F, Zheng J, Deberardinis R J, Mahmoud A I, Olson E N, Schneider J W, Zhang C C, Sadek H A, “The distinct metabolic profile of hematopoietic stem cells reflects their location in a hypoxic niche.” Cell Stem Cell. 2010 Sep. 3; 7(3):380-90.
Spring-Mills E., Stearns S. B., Smith T. H., et al. Immunoreactive hormones in human breast tissues. Surgery 1983; 94(6):946-950.
Sporn M. B., Todaro G. J. Autocrine secretion and malignant transformation of cells. N Engl J Med 1980; 308:487-490.
Surmacz E., Guvakova M. A., Nolan M. K., Nicosia R. F., Sciacca L. Type I insulin-like growth factor receptor function in breast cancer. Breast Cancer Res Treat 1998; 47(3):255-67.
Van der Burg B., de Laat S. W., van Zoelen E. J. J. Mitogenic stimulation of human breast cancer cells in a growth-factor defined medium: synergistic action of insulin and estrogens. In: Brescani F, King R. J. B, Lippman, M. E, Raynaud J. P, eds. Progress in Cancer Research and Therapy, vol. 35: Hormones and Cancer 3. New York, Raven Press, Ltd., 1988: 231-233.
Van Wyk J. J., Graves C. D., Casella S. J., Jacobs, S. Evidence from monoclonal antibody studies that insulin stimulates deoxyribonucleic acid synthesis through the type I somatomedin receptor. J Clin Endocrinol Metab 1985; 61:639-643.
Warburg O. The metabolism of carcinoma cells. J Cancer Res 1925; 9:148-163.
Weber, Wolfgang A., “Use of PET for Monitoring Cancer Therapy and for Predicting Outcome” J Nucl Med. 2005 June; 46(6):983-95.
Winquist R J, Boucher D M, Wood M, Furey B F., “Targeting cancer stem cells for more effective therapies: Taking out cancer's locomotive engine.” Biochem Pharmacol. 2009 Aug. 15; 78(4):326-34.
Wong M., Holdaway I. M. Insulin binding by normal and neoplastic colon tissue. Int J Cancer 1985; 35:335-341.
Workman, Ronald B Jr. and Coleman, R. Edward, “PET/CT Essentials for Clinical Practice” Springer Science+Business Media, LLC, 2006.
Xu R H, Pelicano H, Zhou Y, Carew J S, Feng L, Bhalla K N, Keating M J, Huang P., “Inhibition of glycolysis in cancer cells: a novel strategy to overcome drug resistance associated with mitochondrial respiratory defect and hypoxia.” Cancer Res. 2005 Jan. 15; 65(2):613-21.
Yee D. The insulin-like growth factors and breast cancer—revisited. Breast Cancer Res Treat 1998; 47(3):197-9.
Yoshimasa S., et al. A new approach to the detection of autoantibodies against insulin receptors that inhibit the internalization of insulin into human cells. Diabetes 1984; 33:1051-1054.
Zapf J., Froesch E. R. Insulin-like growth factors/somatomedins: structure, secretion, biological actions and physiological role. Hormone Res 1986; 24:121-130.

We claim:

1. A system for chemotherapy delivery, comprising:
a plurality of slots, wherein each of the plurality of slots is configured to receive a corresponding one of a plurality of cartridges;
a plurality of pumps, wherein each of the plurality of pumps is configured to be connected to the corresponding one of the plurality of cartridges, and the plurality of pumps are configured to pump at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively;
a blood glucose sensor communicatively coupled to the plurality of pumps, and configured to measure a blood glucose level of the patient;
a processor connected to the plurality of pumps and the blood glucose sensor and configured to adjust a delivery property of the at least one drug according to the measured blood glucose level of the patient; and
wherein the plurality of pumps are further configured by the processor to adjust pumping the at least one drug according to the adjusted delivery property.

2. The system of claim 1, wherein one of the plurality of cartridges contains saline, and the system further comprises:
a mixer connected to the plurality of cartridges and the processor and configured to dilute the at least one drug by diluting the insulin, the glucose and the at least one chemotherapeutic drug with the saline according to the adjusted delivery property, wherein the mixer is further configured to deliver the diluted drug to the patient.

3. The system of claim 1, further comprising
an ECG monitoring system communicatively coupled to the processor and configured to monitor heart rhythm of the patient;
wherein the processor is further configured to adjust the delivery property of the at least one drug according to the measured blood glucose level and the heart rhythm of the patient;
wherein the plurality of pumps are further configured by the processor to adjust pumping the at least one drug according to the adjusted delivery property.

4. The system of claim 3, wherein
the ECG monitoring system is further configured to indicate to the processor that an adverse cardiac event is detected for the patient; and
the processor is further configured to instruct the plurality of pumps to return the patient to normal blood glucose levels by changing the amount for pumping for insulin, and/or glucose.

5. The system of claim 1, wherein
the processor is further communicatively connected to a server and configured to download patient data from the sever;
wherein the plurality of pumps are further configured by the processor to adjust pumping the at least one drug according to the patient data.

6. The system of claim 1,
wherein each of the plurality of slots includes a chamber sized to receive the corresponding one of a plurality of cartridges.

7. The system of claim 3, further comprising
a display communicatively coupled to both the ECG monitoring system and the blood glucose sensor, and configured to show a current blood glucose levels and ECG status according to data received from the ECG monitoring system and the blood glucose sensor.

8. The system of claim 1, further comprising
a pressure sensor connected to the plurality of pumps and configured to stop pumping of the plurality of pumps if the pressure sensor detects a blood pressure of the patient is higher than a threshold.

9. The system of claim 1, further comprising
a waste container;
an air detector connected to both the waste container and an output line of the plurality of pumps and configured to remove air from the output line into the waste container.

10. The system of claim 1, further comprising a heater placed in proximity to the plurality of cartridges and configured to remove condensation in the at least one of the plurality of cartridges by heating the at least one of the plurality of cartridges to room temperature.

11. The system of claim 1, wherein the delivery property of the at least one drug comprises the flow rate of drug delivery, volume of drug delivery, treatment time, treatment remaining, medication being administered, remaining medication to administer, medication administered, medication duration, order of drugs to be delivered, wherein the treatment protocol includes at least one of medications and agents to administer and duration and their combination.

12. The system of claim 1, wherein the processor is further configured to control blood sugar levels for an extended period of time by using glucose clamps technique.

13. The system of claim 12, wherein the processor is further configured to control blood sugar levels to induce hypoglycemia for up to two hours by using the glucose clamps technique.

14. The system of claim 1, wherein the plurality of pumps are further configured by the processor to adjust pumping the at least one drug according to the adjusted delivery property before a hypoglycemic therapeutic window induced by a hypoglycemic glucose clamp.

15. The system of claim 1, wherein the plurality of pumps are further configured by the processor to adjust pumping the at least one drug according to the adjusted delivery property before a hypoglycemic therapeutic window induced by a hypoglycemic glucose clamp.

16. A method for chemotherapy delivery, comprising:
receiving, by each of a plurality of slots, a corresponding one of a plurality of cartridges;
pumping, by a plurality of pumps each connected to the corresponding one of the plurality of cartridges, at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively;
measuring, by a blood glucose sensor communicatively coupled to the plurality of pumps, a blood glucose level of the patient;
adjusting, by a processor connected to the plurality of pumps and the blood glucose sensor, delivery property of the at least one drug according to the measured blood glucose level of the patient; and
adjusting, by the plurality of pumps, pumping the at least one drug according to the adjusted delivery property.

17. The method of claim 16, further comprising
diluting, by a mixer connected to the plurality of cartridges and the processor, the at least one drug by diluting the insulin, the glucose and the at least one chemotherapeutic drug according to the adjusted delivery property, and
delivering, by the mixer, the diluted drug to the patient.

18. The method of claim 16, further comprising
monitoring, by an ECG monitoring system communicatively coupled to the processor, heart rhythm of the patient;
adjusting, by the processor, the delivery property of the at least one drug according to the measured blood glucose level and the heart rhythm of the patient; and
adjusting, by the plurality of pumps, pumping the at least one drug according to the adjusted delivery property.

19. The method of claim 18, further comprising
indicating, by the ECG monitoring system, to the processor that an adverse cardiac event is detected for the patient; and
instructing, by the processor to the plurality of pumps, to return the patient to normal blood glucose levels by changing the amount for pumping for insulin, and/or glucose.

20. The method of claim 16, further comprising
downloading, by the processor communicatively connected to a server, patient data from the sever;
adjusting, by the plurality of pumps, pumping of the at least one drug according to the patient data.

21. The method of claim 16,
wherein each of the plurality of pumps includes a chamber sized to receive the corresponding one of a plurality of cartridges.

22. The method of claim 18, further comprising
showing, by a display communicatively coupled to both the ECG monitoring system and the blood glucose sensor, a current blood glucose levels and ECG status according to data received from the ECG monitoring system and the blood glucose sensor.

23. The method of claim 16, further comprising
stopping, by a pressure sensor connected to the plurality of pumps, pumping of the plurality of pumps if the pressure sensor detects a blood pressure of the patient is higher than a threshold.

24. The method of claim 16, further comprising
removing, by an air detector connected to both a waste container and an output line of the plurality of pumps, air from the output line into the waste container.

25. The method of claim 16, further comprising
removing, by a heater placed in proximity to the plurality of cartridges, condensation in the at least one of the plurality of cartridges by heating the at least one of the plurality of cartridges to room temperature.

26. The method of claim 16, wherein the delivery property of the at least one drug comprises the flow rate of drug delivery and volume of drug delivery, treatment time, treatment remaining, medication being administered, remaining medication to administer, medication administered, medication duration, order of drugs to be delivered, wherein the treatment protocol includes at least one of medications and agents to administer and duration and their combination.

27. The method of claim 16, further comprising controlling, by the processor, blood sugar levels for an extended period of time by using glucose clamps technique.

28. The method of claim 27, further comprising controlling, by the processor, blood sugar levels to induce hypoglycemia for up to two hours by using the glucose clamps technique.

29. The method of claim 16, further comprising adjusting by the plurality of pumps, pumping the at least one drug according to the adjusted delivery property before a hypoglycemic therapeutic window induced by a hypoglycemic glucose clamp.

30. The method of claim 16, further comprising adjusting by the plurality of pumps, pumping the at least one drug according to the adjusted delivery property before a hypoglycemic therapeutic window induced by a hypoglycemic glucose clamp.

31. A computer readable storage medium, storing instructions when executed by a processor, cause the computer to perform operations comprising:
controlling, a plurality of pumps each connected to a corresponding one of a plurality of cartridges to pump at least one drug contained in at least one of the plurality of cartridges to a patient according to a treatment protocol, wherein the at least one drug includes insulin, glucose and at least one chemotherapeutic drug, and the plurality of the cartridges are configured to contain insulin, glucose and the at least one chemotherapeutic drug respectively;
controlling a blood glucose sensor communicatively coupled to the plurality of pumps to measure a blood glucose level of the patient;
adjusting delivery property of the at least one drug according to the measured blood glucose level of the patient; and
controlling the plurality of pumps to adjust pumping the at least one drug according to the adjusted delivery property.

32. The computer readable storage medium of claim 31, wherein one of the plurality of cartridges contains saline, and the operations further comprises:
controlling a mixer connected to the plurality of cartridges and the processor to dilute the at least one drug by diluting the insulin, the glucose and the at least one chemotherapeutic drug with the saline according to the adjusted delivery property, and
controlling the mixer to deliver the diluted drug to the patient by adjusting a flow rate and volume of the delivered diluted drug.

33. The computer readable storage medium of claim 31, wherein the operations further comprises
reading heart rhythm of the patient monitor monitored by an ECG monitoring system communicatively coupled to the processor;
adjusting the delivery property of the at least one drug according to the measured blood glucose level and the heart rhythm of the patient;
controlling the plurality of pumps to adjust pumping the at least one drug according to the measured blood glucose level and the heart rhythm of the patient to maintain proper blood sugar level.

34. The computer readable storage medium of claim 31, wherein the operations further comprises
monitoring a detection of an adverse cardiac event for the patient from the ECG monitoring system; and
instructing the plurality of pumps to return patient to normal blood glucose levels by changing the amount for pumping for insulin, and/or glucose.

35. The computer readable storage medium of claim 31, wherein the operations further comprises
downloading patient data from the sever;
controlling the plurality of pumps to adjust pumping the at least one drug according to the patient data; and
storing the adjusted delivery property in a data storage.

36. The computer readable storage medium of claim 31, wherein the operations further comprises
controlling a display communicatively coupled to both the ECG monitoring system and the blood glucose sensor, to show a current blood glucose levels and ECG status according to data received from the ECG monitoring system and the blood glucose sensor.

37. The computer readable storage medium of claim 31, wherein the operations further comprises
controlling an audio communicatively coupled to both the ECG monitoring system and the blood glucose sensor, to output an audio signal indicates a current blood glucose levels and ECG status according to data received from the ECG monitoring system and the blood glucose sensor.

38. The computer readable storage medium of claim 31, wherein the operations further comprises
controlling blood sugar levels for an extended period of time by using glucose clamps technique.

39. The computer readable storage medium of claim 38, wherein the operations further comprises
controlling blood sugar levels to induce hypoglycemia for up to two hours by using the glucose clamps technique.

40. The computer readable storage medium of claim 31, wherein the operations further comprises
controlling a plurality of pumps to adjust pumping the at least one drug according to the adjusted delivery property before a hypoglycemic therapeutic window induced by a hypoglycemic glucose clamp.

41. The computer readable storage medium of claim 31, wherein the operations further comprises
controlling a plurality of pumps to adjust pumping the at least one drug according to the adjusted delivery property before a hypoglycemic therapeutic window induced by a hypoglycemic glucose clamp.
